(19) United States
(12) Patent Application Publication
(10) Pub. No.: US 2015/0125919 A1
(43) Pub. Date: May 7, 2015

(54) MICROORGANISM WITH INCREASED IRON-REGULATED ABC TRANSPORTER ACTIVITY AND METHOD OF PRODUCING HYDROXYCARBOXYLIC ACID BY USING THE MICROORGANISM

(71) Applicant: Samsung Electronics Co., Ltd., Suwon-si (KR)

(72) Inventors: Hwayoung Cho, Hwaseong-si (KR); Jinhwan Park, Suwon-si (KR); Hongsoon Rhee, Hwaseong-si (KR); Kwangmyung Cho, Seongnam-si (KR)

(21) Appl. No.: 14/533,895

(22) Filed: Nov. 5, 2014

(30) Foreign Application Priority Data
Nov. 5, 2013 (KR) 10-2013-0133829
Oct. 28, 2014 (KR) 10-2014-0147637

(51) Int. Cl. C12P 7/52; C07K 14/34; C12N 9/10; C12N 9/04; C12N 9/02

(52) U.S. Cl. CPC C12P 7/52; C12N 9/0006; C12N 9/0008; C12N 9/13; C07K 14/34; C12Y 101/01001; C12Y 102/01016; C12Y 208/03
USPC 435/146; 435/252.33; 435/252.32; 435/252.3

(57) ABSTRACT

A recombinant microorganism having increased iron-regulated ABC transporter activity and increased hydroxycarboxylic acid production, as well as a method of producing a hydroxycarboxylic acid using the recombinant microorganism, and a method of producing the recombinant microorganism.

### RELATED APPLICATIONS

This application claims the benefit of Korean Patent Application No. 10-2013-0133829, filed on Nov. 5, 2013, and Korean Patent Application No. 10-2014-0147637, filed on Oct. 28, 2014, in the Korean Intellectual Property Office, the entire disclosures of which are hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: One 118,997 bytes ASCII (Text) file named “718964_ST25.TXT” created Nov. 4, 2014.

### BACKGROUND

1. Field

The present disclosure relates to microorganisms having increased iron-regulated ABC transporter activity and methods of producing hydroxycarboxylic acid by using the microorganisms.

2. Description of the Related Art

Hydroxycarboxylic acid has a chemical structure including two functional groups, namely, a hydroxy group and a carboxy group, and is capable of being used in various chemical conversions. The hydroxy group and the carboxy group may each independently undergo a chemical reaction or may be transformed into a dimer, an oligomer, or a polymer material through a reaction between them. In the case of a β-hydroxycarboxylic acid, an α or β-unsaturated carboxylic acid may be obtained from a dehydration reaction between the hydroxy group and a nearby hydrogen atom.

The hydroxycarboxylic acid, such as a 4-hydroxybutyric acid or a 3-hydroxypropionic acid, is a useful material that may be used as an intermediate or monomer in the synthesis of a polymer or a pharmaceutical. The 4-hydroxybutyric acid may be used as a biologically important precursor for a C4 compound, such as 1,4-butanediol or γ-butyrolactone, and may also be used as a monomer of polyhydroxy butyrate.

It has been reported that the hydroxycarboxylic acid may be produced by a petrochemical process or a biological method. However, considering the increase in the manufacturing costs due to the increase in the oil price, a method of producing a highly concentrated hydroxycarboxylic acid is needed as a biological method to supplement and supplant a chemical process.

### SUMMARY

Provided is a recombinant microorganism having increased iron-regulated ABC transporter activity and increased hydroxycarboxylic acid production as compared to a parent cell of the microorganism. In one embodiment, the recombinant microorganism has increased expression of a polynucleotide (gene) encoding an iron-regulated ABC transporter, such as a recombinant microorganism having an exogenous gene encoding an iron regulated ABC transporter.

Also provided is a method of producing a hydroxycarboxylic acid using the microorganisms. The method comprises culturing the recombinant microorganism with a carbon source, and recovering the hydroxycarboxylic acid from the culture.

Also provided is a method of providing a recombinant microorganism with improved hydroxycarboxylic acid production, or improving the hydroxycarboxylic acid production of a microorganism. The method comprises introducing an exogenous gene encoding an iron-regulated ABC transporter into a hydroxycarboxylic acid producing microorganism, to provide a recombinant microorganism with improved hydroxycarboxylic acid production.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a cleavage map of a pGSK+ vector;

FIG. 2 is a cleavage map of a pGST1 vector;

FIG. 3 is a cleavage map of a pG3G vector;

FIG. 4 is a cleavage map of a pGS-EX1 vector;

FIG. 5 is a cleavage map of a pEX1-1502 vector;

FIG. 6 is a cleavage map of a pEX1-1503 vector; and

FIG. 7 is a cleavage map of a pEX1-0776 vector.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

As used herein, the term “increase in activity” or “increased activity” may refer to a detectable increase in the activity of a cell, a protein, or an enzyme. In other words, the term “increase in activity” or “increased activity” of a cell, a protein, or an enzyme may refer to a higher activity level of a modified (i.e., genetically engineered) cell, protein, or enzyme than that of a comparable cell of the same type, a protein, or an enzyme without the given genetic modification (e.g., an original or wide-type cell, protein, or enzyme). For example, the activity of the modified or genetically engineered cell, protein, or enzyme may be increased by about 5% or more, about 10% or more, about 15% or more, about 20% or more, about 30% or more, about 50% or more, about 60% or more, about 70% or more, or about 100% or more than the activity of an originally unengineered cell, protein, or enzyme, such as a wild-type cell, protein, or an enzyme. As used herein, the term “unengineered” refers to a cell, protein or enzyme, or gene, etc. that does not have a given specific modification, encompassing but not necessarily limited to an absolutely unmodified (e.g., “wild-type) cell, protein, enzyme, gene, etc. as well as a cell protein, enzyme, gene, etc. that is engineered or recombinant (e.g., not naturally occurring) missing a particular referenced modification. Thus, as compared to a cell comprising a particular modification, an unengineered cell does not contain that particular modification, but may contain other modifications. The activity of particular protein or enzyme in cells may be about 5% or more, about 10% or more, about 15% or more, about 20% or more, about 30% or more, about 50% or more, about 60% or more, about 70% or more, or about 100% or more increased than the activity of a parent cell, e.g., the same protein or enzyme in the originally unengineered cells. The cells with increased activity of a protein or an enzyme may be identified by using methods known in the art, and these cells may have a genetic modification that increases the activity of at least one enzyme or polypeptide as compared with the activity of the cell without having the given genetic modification.

Meanwhile, as used herein, the term “decreased activity” or “decrease in activity” of a cell may refer to an activity level of an enzyme or a polypeptide in a cell that is lower than that measured in a parent cell (e.g., a genetically unengineered cell). In other words, the term “decreased activity” or “decrease in activity” of an enzyme or a polypeptide may refer to an activity level of an isolated enzyme or polypeptide that is lower than that measured in the unengineered (e.g., original or wild-type) enzyme or polypeptide. The “decreased activity” or “decrease in activity” of a cell may also refer to an activity level at which an enzyme or a polypeptide shows no activity. For example, the activity of a modified (i.e., genetically engineered) cell or enzyme in terms of enzymatic conversion of a product from a substrate may be decreased by about 20% or more, about 30% or more, about 40% or more, about 50% or more, about 55% or more, about 60% or more, about 70% or more, about 75% or more, about 80% or more, about 85% or more, about 90% or more, about 95% or more, or about 100% as compared to the activity of an unengineered cell or enzyme, such as a parent cell or a “wild-type” cell or enzyme, in terms of enzymatic conversion. The decreased activity of an enzyme or a cell may be identified by using methods known in the art. The decreased activity includes the case in which the enzyme is inactive or has reduced activity even when the enzyme is expressed as compared with a cell having an unengineered gene, i.e., a parent cell or a wild-type cell, or the case in which the gene encoding the enzyme is not expressed or has reduced expression as compared with the genetically unengineered gene even when the enzyme is expressed. The cells with decreased activity may have a genetic modification that decreases the activity of at least one enzyme or polypeptide as compared with the activity of the cell without having the given genetic modification.

An unengineered cell includes a parent cell. As used herein, the term “parent cell” denotes a cell in a state immediately prior to a particular genetic modification, for example, a cell that serves as a starting material for producing a cell having a genetic modification that increases the activity of a given protein. A parent cell, thus, is a cell without a particular referenced genetic modification, but with the other traits of the genetically modified cell and of the same cell type as the genetically modified cell. Although the “parent cell” does not have the specific referenced genetic modification, the parent cell may be engineered in other respects and, thus, might not be a “wild-type” cell.

As used herein, the term “genetic modification” may refer to introduction of a polynucleotide encoding a polypeptide (i.e., an increase in a copy number of the gene), or substitution, addition, insertion, or deletion of at least one nucleotide with a genetic material of a parent cell, or chemical mutation of a genetic material of a parent cell. In other words, genetic modification may include cases associated with a coding region of a polypeptide or a functional fragment thereof of a polypeptide that is heterologous, homologous, or both heterologous and homologous with a referenced species. Genetic modification may also refer to modification in non-coding regulatory regions that are capable of modifying expression of a gene or an operon, wherein the non-coding regulatory regions include a 5′-non coding sequence and/or a 3′-non coding sequence.

As used herein, the term “disruption” of a gene refers to a genetic modification that decreases expression of the referenced gene. The disruption of the gene may include a case where the referenced gene shows no activity in gene expression thereof (hereinafter, the case is referred to as “inactivation” of the gene), or a case where the referenced gene has a reduced expression level, even when the gene is expressed (hereinafter, the case is referred to as “attenuation” of the gene). In this regard, the inactivation of the gene may refer to not only a case where a functional product of the gene is not expressed, but also a case where the gene is expressed without expressing a functional product, and the attenuation of the gene may refer to a case where expression of the gene is reduced or the activity level of the gene product is reduced. The functional product of the gene may be the gene product of a parent cell or a wild-type cell including a gene with biochemical or physiological function (i.e., enzymatic activity). Thus, the disruption of the gene includes functional disruption of the gene.

The disruption of the gene may be achieved by genetic manipulation including homologous recombination, directed mutagenesis, or molecular evolution. When a cell includes multiple copies of the same gene or two or more paralogs of the gene, at least one of the genes may be disrupted. For example, such genetic modification may be performed by transforming a cell with a vector including a partial sequence of the gene, culturing the transformed cell, disrupting the gene by homologous recombination between the sequence and the endogenous gene, and then by screening the cells of the homologous recombination using a selectable marker.

As used herein, the term “gene” may refer to a nucleic acid fragment expressing a specific protein. The gene may include a regulatory sequence, such as a 5′-non-coding sequence and/or a 3′-non-coding sequence.

As used herein, the term “sequence identity” of nucleic acid or polypeptide may refer to the extent of identity between bases or amino acid residues of sequences after aligning the sequences such that they maximally match in certain comparative regions. The sequence identity is a value calculated by optimally aligning two sequences at certain comparative regions, wherein portions of the sequences at the certain comparative regions may be added or deleted compared to reference sequences. A percentage of sequence identity may be calculated by, for example, comparing two optimally aligned sequences in the entire comparative region, determining the number of locations in which the same amino acids or nucleic acids appear to obtain the number of matched locations, dividing the number of the matched locations by the total number of locations in the comparative region (that is, the size of the range), and multiplying 100 thereto to calculate the percentage of the sequence identity. The percentage of the sequence identity may be calculated by using a known sequence comparison program, and examples of such program include BLASTN (NCBI), CLC Main Workbench (CLC bio), and MegAlign™ (DNASTAR Inc).

Various levels of sequence identity may be used to identify various types of polypeptides or polynucleotides having the same or similar functions. For example, a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100% may be used as a standard.

As used herein, the term “exogenous” gene may refer to a gene or nucleic acid (e.g., encoding a polypeptide or enzyme) that is introduced into a host cell. The exogenous gene may be introduced into a genetic material of the host cell, e.g., a chromosome, or may be introduced as a non-chromosomal genetic material, e.g., a plasmid. In regard to the expression of the encoding nucleic acid, the term “exogenous” indicates a practice of introducing the encoding nucleic acid into an individual in an expressible form. In regard to biosynthetic activity, the term “exogenous” indicates the activity introduced into a host, parent cell. A source of the exogenous gene may be, for example, a homologous or heterologous source. The term “endogenous” denotes a gene (or gene product or biological activity) that is derived from a source that that is of the same genetic origin (e.g., same species) as the host cell. The term “heterologous” denotes a gene (or gene product or activity) that is derived from a different organism, for example, derived from a different cell type or a different species from the recipient. Thus, an exogenous gene or nucleic acid (or gene product or activity) may be heterologous or homologous to a host cell.

In addition, as used herein, the term “genetic engineering” or “genetically engineered” may refer to a practice of performing genetic modification with respect to one or more cells, or a cell resulted from genetic modification. The term “genetically engineered” may be used interchangeably with the term “recombinant”.

According to an aspect of the present invention, provided is a recombinant microorganism having increased iron-regulated ABC transporter activity and increased hydroxycarboxylic acid production as compared to a parent cell of the recombinant microorganism.

The iron-regulated ABC transporter may be a member of ATP-binding cassette transporter (“ABC transporter”) family. For example, the iron-regulated ABC transporter may be a membrane protein for transporting materials through a membrane, or the transporter may be a catalyst for the reaction:

ATP+Fe³⁺(in extracellular space)+H₂O->ADP+Fe³⁺(in cytosol)+phosphate

The transporter may include SufB (e.g., Ncgl1502), SufD (e.g., DNc11503), a member of the ABC-type cobalamin/Fe3+-siderophore transport system, or a combination thereof. Members of the ABC-type cobalamin/Fe3+-siderophore transport system may include ABC-type cobalamin/Fe3+-siderophore transport system, permease (e.g., Ncgl0777 and Ncgl0778), ATPase (Ncgl0779), and periplasmic component (e.g., Ncgl0776). For example, the ABC-type cobalamin/Fe3+-siderophore transport system may include ABC-type cobalamin/Fe3+-siderophore transport system, periplasmic component (e.g., Ncgl0776).

Genes for the iron-regulated ABC transporters, sufB and sufD, may form a part of a suf operon, and participate in an iron metabolism, an assembly of a Fe—S cluster, or oxidative stress response.

The iron-regulated ABC transporter SufB may include an amino acid sequence having a sequence identity of 65% or more, for example, 70% or more, 80% or more, 85% or more, 90% or more, 91′)/0 or more, 92% or more, 93% or more, 94% or more, 95% or more, 96% or more, 97% or more, 98% or more, 99% or more, or 100% to SEQ ID NO: 1. Examples of SufB include NCBI Reference Sequence: NP_{—}600778.1, YP_{—}225849.1, YP_{—}007560762.1, NP_{—}416198.2, YP_{—}489945.1, or YP_{—}006319280.1. The polynucleotide encoding the SufB may have a nucleotide sequence encoding an amino acid sequence having a sequence identity of 95% to the SEQ ID NO: 1 or a nucleotide sequence of SEQ ID NO: 2. The nucleotide sequence coding SufB may be, but is not limited to, Gene ID: 1019532, Gene ID: 62390447, Gene ID: 14794195, Gene ID: 945753, Gene ID: 12931288, or Gene ID: 12955518. The transporter may be derived from a microorganism such as bacteria, yeast, and bacteria, and in greater detail, the microorganism may be Corynebacterium glutamicum, Escherichia coli, or Escherichia blattae.

The iron-regulated ABC transporter SufD may include an amino acid sequence having a sequence identity of 65% or more, for example, 70% or more, 80% or more, 85% or more, 90% or more, 91% or more, 92% or more, 93% or more, 94% or more, 95% or more, 96% or more, 97% or more, 98% or more, 99% or more, or 100% to SEQ ID NO: 3. Examples of SufD include, but are not limited to, NCBI Reference Sequence: NP_{—}600779.1, YP_{—}225848, NP_{—}416196.1, or YP_{—}489943.1. The polynucleotide encoding the SufD may have a nucleotide sequence encoding an amino acid sequence having a sequence identity of 95% to the SEQ ID NO: 3 or a nucleotide sequence of SEQ ID NO: 4. The nucleotide sequence coding SufD may be Gene ID: 1019533, Gene ID: 62390446, Gene ID: 944878, or Gene ID: 12931286. The transporter may be derived from a microorganism such as bacteria, yeast, and bacteria, and in greater detail, the microorganism may be Corynebacterium glutamicum, Escherichia coli, or Escherichia blattae.

The ABC-type cobalamin/Fe3+-siderophore transport system, periplasmic component may include an amino acid sequence having a sequence identity of 65% or more, for example, 70% or more, 80% or more, 85% or more, 90% or more, 91% or more, 92% or more, 93% or more, 94% or more, 95% or more, 96% or more, 97% or more, 98% or more, 99% or more, or 100% to SEQ ID NO: 5. The polynucleotide encoding the transporter may have a nucleotide sequence encoding an amino acid sequence having a sequence identity of 95% or more to the SEQ ID NO: 5 or a nucleotide sequence of SEQ ID NO: 6. The nucleotide sequence may be, but is not limited to, NCgl0776. The transporter may be derived from a microorganism such as bacteria, yeast, and bacteria. More specifically, the microorganism may be Corynebacterium glutamicum, Escherichia coli, or Escherichia blattae.

The increased transporter activity in a cell may increase the production of the hydroxycarboxylic acid. The mode by which production is increased is not particularly limited, but may include increased production of the hydroxycarboxylic acid within the cell and/or increased amount or rate of extracellular secretion of the produced hydroxycarboxylic acid.

In the recombinant microorganism, the SufB, the SufD, and the ABC-type cobalamin/Fe3+-siderophore transport system, periplasmic component may have an amino acid sequence having a sequence identity of 95% or more to SEQ ID NOS: 1, 3, and 5, respectively.

The recombinant microorganism may be subjected to genetic modification to increase activity of the transporter as compared with the activity of the parent cell of the microorganism. The recombinant microorganism may be, for example, a recombinant microorganism in which expression of a polynucleotide encoding an iron-regulated ABC transporter is increased. In other words, the recombinant microorganism may be a recombinant microorganism that expresses or over-expresses the polynucleotide encoding the iron-regulated ABC transporter producing an increased amount of hydroxycarboxylic acid than the microorganism that does not express or over-express the polynucleotide encoding the iron-regulated ABC transporter cultured under the same condition.

In some embodiments, the recombinant microorganism may be a recombinant microorganism transformed with a polynucleotide encoding an iron-regulated ABC transporter that produces an increased amount of hydroxycarboxylic acid compared to a microorganism that is not transformed with the polynucleotide encoding the iron-regulated ABC transporter when cultured under the same conditions. In other words, the recombinant microorganism may include an exogenous gene encoding the transporter, wherein the exogenous gene may encode an amino acid having a sequence identity of 95% or more to each of SEQ ID NOS: 1, 3, and/or 5, respectively. For example, the exogenous gene may a nucleotide sequence having a sequence identity of 95% or more to each of SEQ ID NOS: 2, 4, and/or 6, respectively.

The recombinant microorganism may be genetically engineered to have increased expression of the gene that encodes one or more of the transporter as compared to a genetically unengineered cell (e.g., a parent cell). When the transporter is already present in an active form in a parent cell, the expression of the transporter may be further increased through genetic manipulation. When the transporter is not present in an active form in a wild-type microorganism, the gene encoding the transporter may be introduced into the parent cell to express or over-express the same through genetic manipulation. The genetically unengineered cell refers to a wild-type or a parent cell from which the recombinant microorganism is derived.

The expression or the over-expression of the transporter may be achieved by various methods known to one of ordinary skill in the art. For example, a copy number of the gene may be increased, or a regulatory material such as an inducer or a repressor may be used to increase the expression. The increased copy number may be due to introduction or amplification of the gene. The increased copy number may be achieved by introducing a vector, or an expression cassette, including the transporter gene operably linked a regulatory element into a host cell.

Alternatively, the increased activity of the transporter may be due to changes to the expression regulatory sequence of the gene. The regulatory sequence may be a promoter sequence for gene expression or a transcription terminator sequence. Also, the regulatory sequence may be a sequence that encodes a motif that may affect the gene expression. The motif may be, for example, a secondary structure-stabilizing motif, an RNA destabilizing motif, a splice-activating motif, a polyadenylation motif, an adenine-rich sequence, or an endonuclease recognition region.

Increased hydroxycarboxylic acid may be produced in a recombinant microorganism that produces an increased amount of the iron-regulated ABC transporter as compared to the microorganism that does not express or over-express the polynucleotide encoding the iron-regulated ABC transporter when cultured under the same condition.

The recombinant microorganism may be selected from bacteria, yeast, fungi, or the like, provided the microorganism has (natively or by way of genetic engineering) a biosynthetic pathway for the production of a hydroxycarboxylic acid. The recombinant microorganism may be any one selected from the group consisting of Escherichia sp., rumen bacteria sp., Brevibacterium sp., and Corynebacterium sp., but it is not limited thereto. In one embodiment, the cell may be Corynebacterium sp., such as Corynebacterium glutamicum or Corynebacterium thermoaminogenes. In other embodiments, the microorganism may be E. coli, Brevibacterium flavum, or Brevibacterium lactofermentum.

In an embodiment of the present invention, the hydroxycarboxylic acid may have a chemical structure represented by Formula 1 below:

HO—R¹—COOH [Formula 1]

In Formula 1, R¹ may be a straight or branched C₁-C₂₀ alkyl group, optionally substituted with a phenyl group, and in some embodiments, R¹ may be a straight unsubstituted C₁-C₉ alkyl group. The hydroxycarboxylic acid may be 3-hydroxypropionic acid, 4-hydroxybutyric acid, 3-hydroxy-2-methylpropionic acid, 3-hydroxybutanoic acid, 3-hydroxy-2-methylbutanoic acid, 3-hydroxy-2-methylpentanoic acid, 3-hydroxy-3-methylbutanoic acid, 2,3-dimethyl-3-hydroxybutanoic acid, 3-hydroxy-3-phenylpropionic acid, or a combination thereof.

The recombinant microorganism may be a type (species) that naturally produces hydroxycarboxylic acid, or may be a microorganism that has been genetically engineered to produce the hydroxycarboxylic acid. For example, when the hydroxycarboxylic acid is 4-hydroxybutyric acid, 4-hydroxybutyric acid may be produced by biological transformation of succinic acid or alpha-ketoglutarate. In the case of using succinic acid as a starting material in biosynthesis of 4-hydroxybutyric acid, 4-hydroxybutyric acid may be synthesized by transforming succinic acid into succinyl-CoA, succinyl-CoA into succinic semialdehyde, and succinic semialdehyde into 4-hydroxybutyric acid. Alternatively, in the case of using α-ketoglutarate as a starting material in biosynthesis of 4-hydroxybutyric acid, 4-hydroxybutyric acid may be synthesized by transforming α-ketoglutarate into succinic semialdehyde, and succinic semialdehyde into 4-hydroxybutyric acid.

The recombinant microorganism may include at least one of an exogenous gene encoding an enzyme that catalyzes conversion of succinic acid to succinyl-CoA, an exogenous gene encoding an enzyme that catalyzes conversion of succinyl-CoA to succinic semialdehyde, and an exogenous gene encoding an enzyme that catalyzes conversion of succinic semialdehyde to 4-hydroxybutyric acid.

The enzyme catalyzing conversion of succinic acid to succinyl-CoA may be categorized as EC.2.8.3.- or EC.2.8.3.8. The enzyme may be derived from Clostridium kluyveri, Corynebacterium kroppenstedtii, Corynebacterium glutamicum, Pseudomonas protegens, or the like. The enzyme may include an amino acid sequence having a sequence identity of 65% or more, for example, 70% or more, 80% or more, 85% or more, 90% or more, 91% or more, 92% or more, 93% or more, 94% or more, 95% or more, 96% or more, 97% or more, 98% or more, 99% or more, or 100% to SEQ ID NO: 7, 9, or 11. The enzyme having an amino acid sequence of SEQ ID NO: 7 may be succinyl-CoA:coenzyme A transferase (Cat1) derived from Clostridium kluyveri. The enzymes each having an amino acid sequence of SEQ ID NOS: 9 and 11 may be acetyl-CoA hydrolase (ActA or ActB) derived from Corynebacterium glutamicum, and these enzymes may include an amino acid sequence of GenBank No: AAA92346.1 (J. Bacteriol. 178:871-880(1996)), GenBank NO: ACR17185.1 (J. Biotechnol. 136 (1-2), 22-30 (2008)), NCBI YP_{—}007997424.1, or the like. The exogenous gene encoding the enzyme that catalyzes conversion of succinic acid to succinyl-CoA may include a nucleotide sequence having a sequence identity of 95% or more to SEQ ID NO: 8, 10, and 12, respectively. The recombinant microorganism may include two or more exogenous genes encoding the enzyme that catalyzes conversion of succinic acid to succinyl-CoA.

The enzyme catalyzing conversion of succinyl-CoA to succinic semialdehyde may be categorized as EC.1.2.1.16. The enzyme may be CoA-dependent succinate semialdehyde dehydrogenase (hereinafter, referred to as “CoA-dependent SSADH”). The enzyme may include an amino acid sequence having a sequence identity of 65% or more, for example, 70% or more, 80% or more, 85% or more, 90% or more, 91% or more, 92% or more, 93% or more, 94% or more, 95% or more, 96% or more, 97% or more, 98% or more, 99% or more, or 100% to SEQ ID NO: 13. Alternatively, the enzyme may include an amino acid sequence of Accession No: P38947.1 (GI NO: 730847), NP_{—}904963.1 (GI NO: 34540484), or the like. The CoA-dependent SSADH may be derived from Clostridium kluyveri, Porphyromonas gingivalis, or the like. The polynucleotide encoding the CoA-dependent SSADH may include a nucleotide sequence encoding an amino acid having a sequence identity of 95% or more to SEQ ID NO: 13. The polynucleotide may have a nucleotide sequence with a sequence identity of 95% or more to SEQ ID NO: 14 (sucD).

The enzyme catalyzing conversion of succinic semialdehyde to 4-hydroxybutyric acid may be categorized as EC.1.1.1.1. The enzyme may be an alcohol dehydrogenase or 4-hydroxybutyric acid dehydrogenase (4hbd). The enzyme may include an amino acid sequence having a sequence identity of 65% or more, for example, 70% or more, 80% or more, 85% or more, 90% or more, 91′)/0 or more, 92% or more, 93% or more, 94% or more, 95% or more, 96% or more, 97% or more, 98% or more, 99% or more, or 100% to SEQ ID NO: 15 (Porphyromonas gingivalis). Alternatively, the enzyme may include an amino acid sequence of NCBI Reference Sequence: NP_{—}904964.1, YP_{—}726053.1, EDK35022.1, or Q94B07. The 4-hydroxybutyric acid dehydrogenase may be derived from Porphyromonas gingivalis, Ralstonia eutropha, Clostridium kluyveri, or Arabidopsis thaliana. The polynucleotide encoding the 4-hydroxybutyric acid dehydrogenase may include a nucleotide sequence encoding an amino acid sequence having a sequence identity of 95% or more to SEQ ID NO: 15. The polynucleotide may have a nucleotide sequence with a sequence identity of 95% or more to SEQ ID NO: 16 (4hbd). Also, a nucleotide sequence encoding the 4-hydroxybutyric acid dehydrogenase may be a nucleotide sequence of Gene ID No: 2552693, 113867564, 146348486, or 75249805.

The recombinant microorganism may include at least one of an exogenous gene encoding an enzyme that catalyzes conversion of α-ketoglutarate into succinic semialdehyde and an exogenous gene encoding an enzyme that catalyzes conversion of succinic semialdehyde into 4-hydroxybutyric acid.

The enzyme catalyzing conversion of α-ketoglutarate into succinic semialdehyde may be categorized as EC.4.1.1.71. The enzyme may be α-ketoglutarate decarboxylase, and the α-ketoglutarate decarboxylase may include an amino acid sequence having a sequence identity of 65% or more, for example, 70% or more, 80% or more, 85% or more, 90% or more, 91% or more, 92% or more, 93% or more, 94% or more, 95% or more, 96% or more, 97% or more, 98% or more, 99% or more, or 100% to SEQ ID NO: 17 or 18. A gene encoding the α-ketoglutarate decarboxylase may be, for example, a nucleotide sequence encoding an amino acid sequence having a sequence identity of 95% or more to each of SEQ ID NO: 17 or 18, or a nucleotide sequence of SEQ ID NO: 19 or 20. The α-ketoglutarate decarboxylase may be derived from, for example, Corynebacterium sp., Rhodococcus sp., Mycobacterium sp., or Escherichia sp. A gene encoding the α-ketoglutarate decarboxylase may be, for example, Corynebacterium glutamicum, Corynebacterium callunae, Corynebacterium efficiens, Corynebacterium ulcerans, Corynebacterium halotolerans, Corynebacterium pseudotuberculosis, Corynebacterium durum, Corynebacterium striatum, Rhodococcus pyridinivorans, Rhodococcus ruber, Mycobacterium abscessus, Mycobacterium smegmatis, Escherichia coli, Escherichia fergusonii, or a combination thereof.

The exogenous gene encoding the enzyme that catalyzes conversion of succinic semialdehyde into 4-hydroxybutyric acid may be defined the same as described above.

The exogenous genes may be introduced into the recombinant microorganism in an expressible form, and accordingly, may be over-expressed compared to gene expression in an unengineered microorganism or a parent cell.

The hydroxycarboxylic acid produced from the recombinant microorganism may be converted into various useful materials. In the case of the 4-hydroxybutyric acid, the hydroxycarboxylic acid may be converted into gamma-butyrolactone (GBL), 1,4-butanediol, poly-4-hydroxybutyric acid, tetrahydrofuran (THF), or N-methylpyrrolidone (NMP).

In addition, the recombinant microorganism may have a genetic element that inhibiting a pathway reducing the production of the hydroxycarboxylic acid. In this regard, the recombinant microorganism may have disrupted activity in converting succinic semialdehyde into succinic acid. That is, the recombinant microorganism may have a gene encoding succinic semialdehyde dehydrogenase (SSADH) removed or disrupted. The succinic semialdehyde dehydrogenase (SSADH) may catalyze a reaction that converts succinic semialdehyde into succinic acid. The SSADH may be an enzyme categorized as EC.1.2.1.16 or EC.1.2.1.39. The SSADH may be NAD+ or NADP+ dependent. The SSADH may include an amino acid sequence having a sequence identity of 65% or more, for example, 70% or more, 80% or more, 85% or more, 90% or more, 91% or more, 92% or more, 93% or more, 94% or more, 95% or more, 96% or more, 97% or more, 98% or more, 99% or more, or 100% to SEQ ID NO: 21, SEQ ID NO: 22, and SEQ ID NO: 23, respectively. The gene that encodes the SSADH may include a polynucleotide that encodes an amino acid sequence having a sequence identity of 95% or more to SEQ ID NO: 21, SEQ ID NO: 22, and SEQ ID NO: 23, respectively, or the polynucleotide may have a nucleotide sequence a sequence identity of 95% or more to SEQ ID NO: 24, SEQ ID NO: 25, or SEQ ID NO: 26. The microorganism may have at least one of a polynucleotide having a nucleotide sequence of SEQ ID NO: 24, a polynucleotide having a nucleotide sequence of SEQ ID NO: 25, and a polynucleotide having a nucleotide sequence of SEQ ID NO: 26 disrupted or removed. The microorganism may have a polynucleotide having a nucleotide sequence of SEQ ID NO: 24 or a polynucleotide having a nucleotide sequence of SEQ ID NO: 25 disrupted or removed. The microorganism may have a polynucleotide having a nucleotide sequence of SEQ ID NO: 24 and a polynucleotide having a nucleotide sequence of SEQ ID NO: 25 disrupted or removed. The microorganism may have a polynucleotide having a nucleotide sequence of SEQ ID NO: 24, a polynucleotide having a nucleotide sequence of SEQ ID NO: 25, and a polynucleotide having a nucleotide sequence of SEQ ID NO: 26 disrupted or removed.

The recombinant microorganism may have a pathway for converting pyruvate into lactate disrupted. That is, the recombinant microorganism may have a gene encoding an enzyme that catalyzes the conversion of pyruvate into lactate disrupted. The enzyme catalyzing the conversion of pyruvate into lactate may be lactate dehydrogenase (LDH) categorized as EC.1.1.1.27 or EC.1.1.1.28. The LDH may be NAD(P)H dependent, and in addition, may function in D-lactate and/or L-lactate. The LDH may include an amino acid sequence having a sequence identity of 65% or more, for example, 70% or more, 80% or more, 85% or more, 90% or more, 91′)/0 or more, 92% or more, 93% or more, 94% or more, 95% or more, 96% or more, 97% or more, 98% or more, 99% or more, or 100% to an amino acid sequence of SEQ ID NO: 27. The recombinant microorganism may have a gene encoding the LDH that is disrupted or removed. Here, the gene encoding the LDH may have a nucleotide sequence encoding an amino acid sequence having a sequence identity of 95% or more to the SEQ ID NO: 27, or a nucleotide sequence with a sequence identity of 95% or more to SEQ ID NO:89 (Ncgl2810).

The recombinant microorganism may be, for example, a recombinant microorganism that produces 4-hydroxybutyric acid, including at least one of a polynucleotide encoding the iron-regulated ABC transporter, a polynucleotide encoding the succinyl-CoA:coenzyme A transferase, and a polynucleotide encoding the CoA-dependent SSADH. In some embodiments, the recombinant microorganism may further include a polynucleotide encoding the 4hbd. The recombinant microorganism may have at least one of genes encoding the LDH and the SSADH that are disrupted.

Another aspect of the present invention provides a method of producing hydroxycarboxylic acid comprising culturing the microorganism that produces the hydroxycarboxylic acid as described above; and recovering the hydroxycarboxylic acid from the culture.

The culturing may be performed in a suitable medium under suitable culturing conditions known in the art. One of ordinary skill in the art may suitably change a culture medium and culturing conditions according to the microorganism selected. A culturing method may be batch culturing, continuous culturing, fed-batch culturing, or a combination thereof.

The culture medium may include various carbon sources, nitrogen sources, and trace elements.

The carbon source may be, for example, carbohydrate such as glucose, sucrose, lactose, fructose, maltose, starch, or cellulose; fat such as soybean oil, sunflower oil, castor oil, or coconut oil; fatty acid such as palmitic acid, stearic acid, linoleic acid; alcohol such as glycerol or ethanol; organic acid such as acetic acid, or a combination thereof. The culturing may be performed by having glucose as the carbon source. The nitrogen source may be an organic nitrogen source such as peptone, yeast extract, beef stock, malt extract, corn steep liquor (CSL), or soybean flour, or an inorganic nitrogen source such as urea, ammonium sulfate, ammonium chloride, ammonium phosphate, ammonium carbonate, and ammonium nitrate, or a combination thereof. The culture medium is a supply source of phosphorus and may include, for example, potassium dihydrogen phosphate, dipotassium phosphate, and corresponding sodium-containing salt thereof, and a metal salt such as magnesium sulfate or iron sulfate. Also, amino acid, vitamin, a suitable precursor, or the like may be included in the culture medium. The culture medium or individual component may be added to a culture medium solution in a batch, fed-batch, or continuous manner.

Also, pH of the culture medium solution may not be adjusted or may be adjusted by adding a compound such as ammonium hydroxide, potassium hydroxide, ammonia, phosphoric acid, and sulfuric acid to the culture medium solution by using a suitable method during the culturing process. Also, an antifoaming agent such as fatty acid polyglycol ester may be used during the culturing process to inhibit the generation of bubbles.

The culturing process may be performed in a suitable oxygen condition for the production of the hydroxycarboxylic acid. The cells may be cultured in an aerobic condition for the cell proliferation. Afterwards, in order to produce the hydroxycarboxylic acid, the cells may be cultured in a microaerobic or anaerobic condition. The term “anaerobic condition” as used herein refers to a condition without oxygen. The term “microaerobic condition” as used herein in terms of cell culture or growth conditions refers to a condition in which a concentration of dissolved oxygen in a medium is maintained greater than 0% and below about 10% of the concentration of dissolved oxygen in an oxygen-saturated liquid medium of the same type. The microaerobic condition may also refer to a condition in which cells are grown or present in a resting state within a sealed chamber held in an atmosphere with an oxygen content of less than 1%. The concentration of oxygen may be maintained by, for example, sparging a culture with a N₂/CO₂ mixture or other suitable non-oxygen gas. Oxygen conditions may also include a concentration of dissolved oxygen is maintained in a range of about 0% to about 10%, for example, about 0% to about 8%, about 0% to about 6%, about 0% to about 4%, about 0% to about 2%.

The hydroxycarboxylic acid produced by the recombinant microorganism may be secreted from the cell and then recovered from the culture medium, and may additionally be separated from the culture medium. The separation of the hydroxycarboxylic acid from the culture medium solution may be performed by a separation and purification method known in the art. The recovery may be performed by centrifugation, chromatography, extraction, filtration, precipitation, or a combination thereof.

The hydroxycarboxylic acid produced by the method described above may be chemically converted to a compound that is structurally related thereto. For example, 4-hydroxybutyric acid may be reacted in the presence of a strong acid at a temperature of about 100° C. to about 200° C. and then distilled to obtain gamma-butyrolactone (GBL). The obtained GBL may be converted into N-methylpyrrolidone (NMP) by amination using an aminating agent, for example, methylamine. Also, the GBL may be selectively converted into tetrahydrofuran (THF), 1,4-butanediol, or butanol by hydrogenation reaction using a metal-containing catalyst such as a catalyst including copper (Cu), ruthenium (Ru), and palladium (Pd). Also, the 4-hydroxybutyric acid may be biologically converted into poly-4-hydroxybutyric acid. The biological conversion may be performed by polyhydroxyalkanoate synthase, 4HB-coenzyme A:coenzyme A transferase, or a combination thereof.

According to an aspect of the present invention, provided is a method of improving the hydroxycarboxylic acid production of a microorganism, the method comprising introducing an exogenous gene encoding an iron-regulated ABC transporter into a hydroxycarboxylic acid producing microorganism, thereby improving hydroxycarboxylic acid production in the microorganism.

In some embodiments, the microorganism having increased activity of the iron-regulated ABC transporters may be used for the production of the hydroxycarboxylic acid.

In some embodiments, the hydroxycarboxylic acid may be produced in an effective manner by using the method described above.

Hereinafter, the present invention is described in greater detail with reference to embodiments. However, the embodiments are for illustrative purposes only and do not limit the scope of the present invention.

### Example 1

### Preparing a Strain Having a L-Lactate Dehydrogenase (ldh) Gene Removed Therefrom

(1) Manufacturing a Replacement Vector

L-lactate dehydrogenase (ldh) gene of Corynebacterium glutamicum (CGL) ATCC 13032 was inactivated by homologous recombination using a pK19 mobsacB (ATCC 87098) vector.

Two homologous regions for removing the ldh gene were obtained by PCR amplification using a genomic DNA of Corynebacterium glutamicum ATCC 13032 as a template. The homologous regions were regions upstream and downstream of the ldh gene, which were obtained by PCR amplification using a primer set of ldhA_{—}5′_HindIII (SEQ ID NO: 28) and ldhA_up_{—}3′_XhoI (SEQ ID NO: 29) and a primer set of ldhA_dn_{—}5′_XhoI (SEQ ID NO: 30) and ldhA_{—}3′_EcoRI (SEQ ID NO: 31). The PCR amplification was performed by repeating the processes of denaturing at a temperature of 95° C. for 30 seconds, annealing at a temperature of 55° C. for 30 seconds, and elongation at a temperature of 72° C. for 30 seconds. All PCR amplifications were performed in the same manner.

The amplification product obtained therefrom was cloned at HindIII and EcoRI restriction sites of the pK19 mobsacB vector to manufacture a pK19_Δldh vector.

(2) Preparing a CGL (Δldh) Strain

Corynebacterium glutamicum ATCC 13032 was electroporated to introduce the pK19_Δldh vector therein. The introduced strain was smeared on 25 μg/ml of a kanamycin-containing LBHIS culture medium and then cultured at a temperature of 30° C. LBHIS culture medium includes 18.5 g/L of brain-heart infusion broth, 0.5 M sorbitol, 5 g/L of bacto-tryptone, 2.5 g/L of bacto-yeast extract, 5 g/L of NaCl, and 18 g/L of bacto-agar. Hereinafter, the composition of the LBHIS culture medium is as described above. The colony formed therefrom was smeared on a LB-sucrose culture medium and then cultured at a temperature of 30° C., followed by selection of colonies in which double cross-over occurred. Genomic DNA was separated from the selected colonies, and a primer set of ldhA up (SEQ ID NO: 32) and ldhA down (SEQ ID NO: 33) was used to confirm deletion of the ldh gene through PCR. As a result, a CGL (Δldh) strain was obtained.

### Example 2

### Deletion of CoA-Dependent Succinic Semialdehyde Dehydrogenase Gene

(1) Manufacturing a Replacement Vector

A CoA-dependent succinic semialdehyde dehydrogenase (ssadh) gene of Corynebacterium glutamicum ATCC 13032, which encodes an enzyme catalyzing the conversion of succinic semialdehyde into succinic acid, was inactivated by homologous recombination using a pK19 mobsacB vector.

Homologous regions for removing NCgl0049 (SEQ ID NO: 24), NCgl0463 (SEQ ID NO: 25), and NCgl2619 (SEQ ID NO: 26), which are CoA-dependent ssadh genes, were obtained by PCR amplification using genomic DNA of Corynebacterium glutamicum ATCC 13032 as a template.

Two homologous regions for removing the NCgl0049 gene (regions upstream and downstream of the gene) were obtained by PCR amplification using a primer set of 0049-1 for (SEQ ID NO: 34) and 0049-1 rev (SEQ ID NO: 35), and a primer set of 0049-2 for (SEQ ID NO: 36) and 0049-2 rev (SEQ ID NO: 37). The amplification product obtained therefrom was cloned at HindIII and PstI restriction sites of the pK19 mobsacB vector to manufacture a pK19_Δn0049 vector.

Two homologous regions for removing the NCgl0463 gene (regions upstream and downstream of the gene) were obtained by PCR amplification using a primer set of 0463-1 for (SEQ ID NO: 38) and 0463-1 rev (SEQ ID NO: 39) and a primer set of 0463-2 for (SEQ ID NO: 40) and 0463-2 rev (SEQ ID NO: 41). The amplification product obtained therefrom was cloned at HindIII and PstI restriction sites of the pK19 mobsacB vector to manufacture a pK19_Δn0463 vector.

Two homologous regions for removing the NCgl2619 gene (regions upstream and downstream of the gene) were obtained by PCR amplification using a primer set of 2619-1 for (SEQ ID NO: 42) and 2619-1 rev (SEQ ID NO: 43) and a primer set of 2619-2 for (SEQ ID NO: 44) and 2619-2 rev (SEQ ID NO: 45). The amplification product obtained therefrom was cloned at HindIII and PstI restriction sites of the pK19 mobsacB vector to manufacture a pK19_Δn2619 vector.

(2) Preparing a CGL (Δldh ΔgD³) Strain

pK19_Δn0049, pK19_Δn0463, and pK19_Δn2619 vectors were sequentially introduced into the CGL (Δldh) strain prepared in Example 1 by electroporation. The introduced strain was smeared on 25 μg/ml of kanamycin-containing LBHIS culture medium and then cultured at a temperature of 30° C. The colony formed therefrom was smeared on a LB-sucrose culture medium and then cultured at a temperature of 30° C., followed by selection of the colonies in which double-crossing occurred. Genomic DNA was separated from the selected colonies, and a primer set of 0049for (SEQ ID NO: 46) and 0049rev (SEQ ID NO: 47), a primer set of 0463for (SEQ ID NO: 48) and 0463rev (SEQ ID NO: 49), or a primer set of 2619for (SEQ ID NO: 50) and 2619rev (SEQ ID NO: 51) was used to confirm the deletion of NCgl0049, NCgl0463 and NCgl2619 by using PCR. As a result, a CGL (Δldh ΔgD³) strain was obtained. Table 1 below shows strain names and gene types prepared in the present invention, wherein D003 is CGL (Δldh, ΔgD³, G3G).

TABLE 1

| Strain name | Gene type |
|---|---|
| CGL (Δldh ΔgD³) | C. glutamicum ATCC 13032 |
| | (Δldh ΔNCgl0049 ΔNCgl0463 ΔNCgl2619) |
| D003 | C. glutamicum ATCC13032 |
| | (Δldh, ΔNCgl0049, ΔNCgl0463, ΔNCgl2619, |
| | ΔgapA::cat1, sucD, 4hbD) |
| D003: pEX1-1502 | C. glutamicum ATCC13032 |
| | (Δldh, ΔNCgl0049, ΔNCgl0463, ΔNCgl2619, |
| | gapA::cat1, sucD, 4hbD pEX1-1502) |
| D003: pEX1-1503 | C. glutamicum ATCC13032 |
| | (Δldh, ΔNCgl0049, ΔNCgl0463, ΔNCgl2619, |
| | gapA::cat1, sucD, 4hbD pEX1-1503) |

### Example 3

### Introduction of cat1, sucD, and 4hbD Genes

(1) Manufacturing a pGST1 Vector

To express cat1, sucD, and 4hbD genes from a vector, a cat1, sucD, and 4hbD gene expression vector, pG3G, was manufactured based on a pGST1 vector. The pGST1 vector was manufactured according to the method described below.

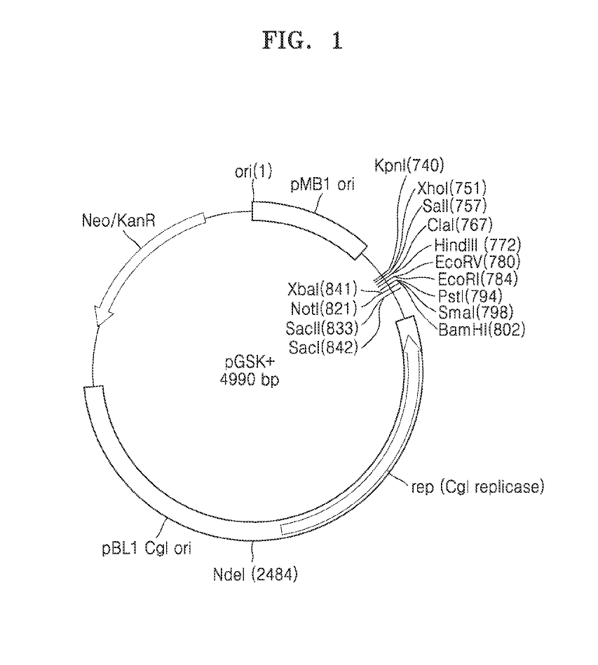

Phusion High-Fidelity DNA Polymerase (cat.# M0530, a product of New England Biolabs) was used to obtain four PCR products. PCR (Cgl replication origin including Cgl replicase gene) was performed by using (i) a C. glutamicum promoter screening vector, pET2 (GenBank accession number: AJ885178.1, 7513 bp), as a template and a primer sequence of MD-616 (SEQ ID NO: 52) and MD-618 (SEQ ID NO: 53); (ii) by using the C. glutamicum promoter screening vector, pET2, as a template and a primer sequence of MD-615 (SEQ ID NO: 54) and MD-617 (SEQ ID NO: 55) (E. coli replication origin); (iii) by using a mammalian fluorescence protein expression vector, pEGFP-C1 (a product of Clontech), as a template and a primer sequence of MD-619 (SEQ ID NO: 56) and MD-620 (SEQ ID NO: 57) (Kanamycin resistant neo gene (NptII)); and (iv) by using an E. coli cloning vector, pBluescriptII SK+, as a template and a primer sequence of LacZa-NR (SEQ ID NO: 58) and a primer sequence of MD-404 (SEQ ID NO: 59) (Multiple cloning site: MCS). All four PCR products, i.e., 3010 bp, 854 bp, 809 bp, and 385 bp, were ligated to a linearized pUC19 vector available from In-Fusion EcoDry PCR Cloning Kit (cat.#639690, available from Clontech), to obtain a circular plasmid. It was confirmed that the 4 genes were ligated to the linearized pUC19 vector. The vector was named pGSK+(SEQ ID NO: 90). FIG. 1 is a cleavage map of the pGSK+ vector.

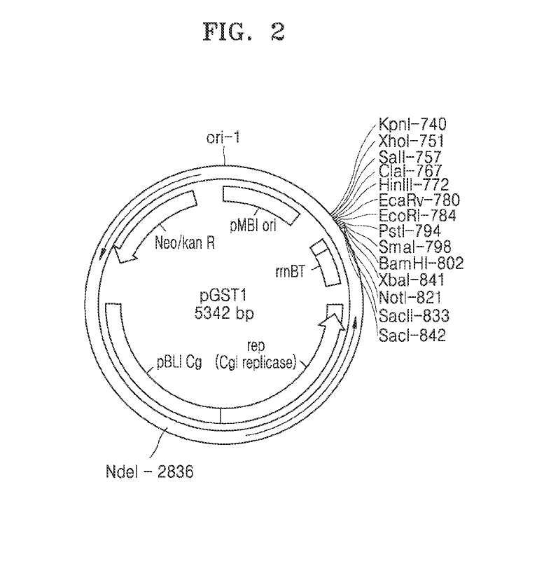

To manufacture a C. glutamicum shuttle vector including a transcription terminator and a 3′ untranslated region (UTR) of the pSGK+ vector, a 3′ UTR of C. glutamicum gltA (NCgl0795) and a rho-independent terminator of rrnB of E. coli rrnB were inserted into the pGSK+ vector. PCR was performed by using a C. glutamicum (ATCC13032) genomic DNA as a template and using a primer sequence of MD-627 (SEQ ID NO: 60) and MD-628 (SEQ ID NO: 61) to obtain a 108 bp PCR fragment of gltA 3′ UTR. Genomic DNA of E. coli (MG1655) was used as a template and a primer sequence of MD-629 (SEQ ID NO: 62) and MD-630 (SEQ ID NO: 63) was used to obtain a 292 bp PCR product of rrnB transcription terminator. Two amplified fragments were inserted into pGSK+ cleaved by SacI using In-Fusion EcoDry PCR Cloning Kit (cat.#639690, a product of Clontech). The cloned vector was introduced into One Shot TOP10 Chemically Competent Cell (cat.# C4040-06, a product of Invitrogen) and then cultured in 25 ug/ml of kanamycin-containing LB culture medium, followed by selection of growth colonies therefrom. A vector was recovered from the selected colonies for whole sequence analysis. The vector was named pGST1 (SEQ ID NO: 64). FIG. 2 is a cleavage map of the pGST1 vector. In FIG. 2, “rrnBT” denotes a gltA 3′-UTR and a transcription terminator of rrn.

(2) Manufacturing a pG3G Vector

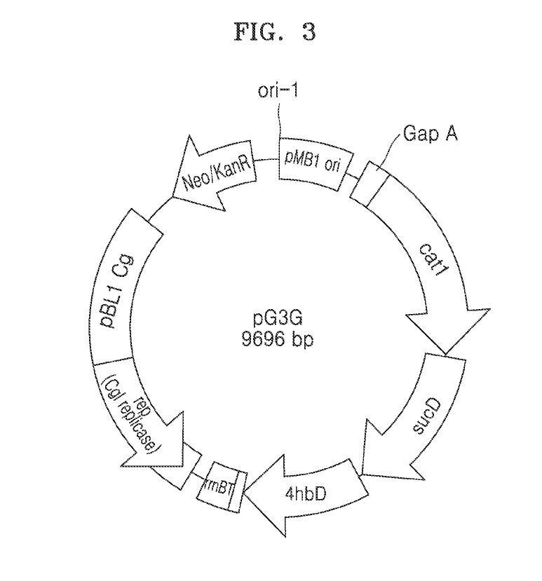

As a template for PCR amplification of the three types of genes described above, a gene cassette (SEQ ID NO: 65) sequentially including gapA promoter, and cat1, sucD, 4hbD, and cat2 genes was synthesized. In the cassette, the gapA promoter, cat1, sucD, and 4hbD gene sequences were obtained by PCR amplification using a primer set of 3G up (SEQ ID NO: 66) and 3G down (SEQ ID NO: 67). The amplification product obtained therefrom was cloned at PstI and XbaI restriction sites of pGST1 vector using an In-fusion HD cloning kit (#PT5162-1, a product of Clontech) to manufacture an expression vector pG3G. FIG. 3 is a cleavage map of the pG3G vector. The gapA promoter was a promoter of gapA gene (NCgl1526) derived from Corynebacterium glutamicum.

(3) Manufacturing a pK19 gapA::3G Vector

To insert the three types of genes into a genome, a vector, pK19 gapA::3G, for inserting cat1, sucD and 4hbD genes was manufactured based on pK19 mobsacB.

Two homologous regions for homologous recombination were obtained by PCR amplification using a genomic DNA of Corynebacterium glutamicum ATCC 13032 as a template, and using each of a primer set of ncgl0049_up F (SEQ ID NO: 68) and ncgl0049_up R (SEQ ID NO: 69), and a primer set of ncgl0049_down F (SEQ ID NO: 70) and ncgl0049_down R (SEQ ID NO: 71).

A fragment including gapA promoter and the cat1, sucD, and 4hbD genes was obtained by PCR amplification using a pG3G vector as a template and using a primer set of pG3G F (SEQ ID NO: 72) and pG3G R (SEQ ID NO: 73).

In the HindIII and EcoRI restriction sites of the pK19 mobsacB vector, the amplified product was cloned to locate the fragment including the gapA promoter and cat1, sucD, and 4hbD genes between the two homologous regions by using an In-fusion HD cloning kit (#PT5162-1, a product of Clontech), to thereby manufacture a pK19 gapA::3G vector.

(4) Preparing a D003 Strain

The pK19 gapA::3G vector was introduced into the CGL (Δldh ΔgD³) strain prepared in Example 1 by electroporation. The introduced strain was smeared on 25 μg/ml of a kanamycin-containing LBHIS culture medium and then cultured at a temperature of 30° C. The colony was smeared on a LB-sucrose culture medium and then cultured at a temperature of 30° C., followed by selection of colonies in which double cross-over occurred. A genomic DNA was separated from the selected colonies, and introduction of the cat1, sucD and 4hbD genes was confirmed by PCR amplification using a primer set of cat1-seq1F (SEQ ID NO: 74) and cat1-seq2R (SEQ ID NO: 75), a primer set of sucD-seq1F (SEQ ID NO: 76) and sucD-seq2R (SEQ ID NO: 77), or a primer set of 4hbD-seq1F (SEQ ID NO: 78) and 4hbD-seq2R (SEQ ID NO: 79), respectively. As a result, a D003 strain, namely, CGL (Δldh, ΔgD³, G3G) was obtained.

### Example 4

### Introduction of sufD, sufB, and Ncgl0776

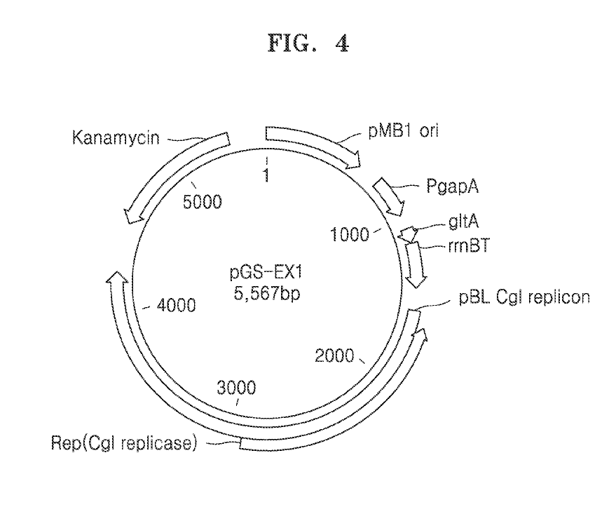

As a plasmid vector for expressing sufD, sufB, and NCgl0776 genes, a pGS-Ex1 (SEQ ID NO: 80) vector including a gapA promoter was manufactured as follows. A Corynebacterium glutamicum-derived promoter of a gapA gene (NCgl1526) was obtained by PCR amplification using a genomic DNA of Corynebacterium glutamicum ATCC 13032 as a template, and a primer set of MD-625 (SEQ ID NO: 81) and MD-626 (SEQ ID NO: 82). The amplified product was cloned such that two DNA fragments of 270 bp each obtained from the PCR reaction were located at KpnI and XhoI restriction sites of the pGST1 vector manufactured in Example 3 to provide the pGS-Ex1 vector. Cloning was performed using an In-fusion HD cloning kit (#PT5162-1, a product of Clontech). FIG. 4 is a cleavage map of the pGS-EX1 vector.

(1) Manufacturing pEX1-1502, pEX1-1503, and pEX1-0776 Vectors

To express NCgl 1502 (sufD), NCgl 1503 (sufB), and Ncgl 0776 genes from a vector, NCgl 1502 (sufD), NCgl 1503 (sufB), and Ncgl 0776 gene expression vectors of pEX1-1502, pEX1-1503, and pEX1-0776 were each manufactured based on the pGS-Ex1 vector (SEQ ID NO: 80).

For PCR amplification of the NCgl 1502, NCgl 1503, and NCgl 0776 genes, genomic DNA of Corynebacterium glutamicum ATCC 13032 was used as a template with a primer sets as follows:
NCgl 1502: 1502-up (SEQ ID NO: 83) and 1502-dn (SEQ ID NO: 84)
NCgl 1503: 1503-up (SEQ ID NO: 85) and 1503-dn (SEQ ID NO: 86)
NCgl 0776: 0776-up (SEQ ID NO: 87) and 0776-dn (SEQ ID NO: 88).

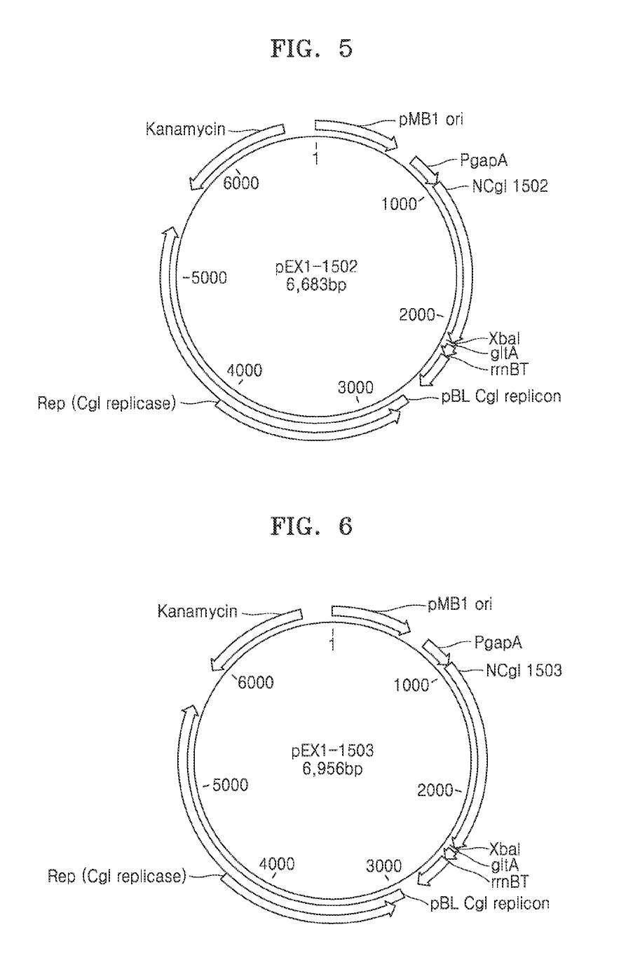

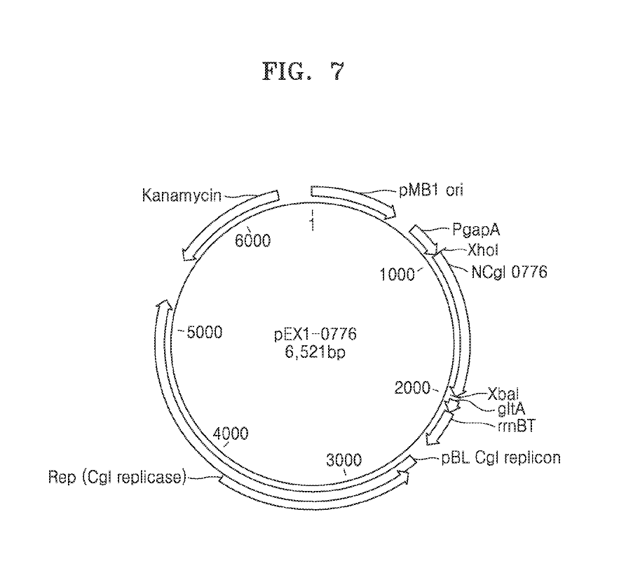

The amplification products obtained therefrom were cloned at XhoI and XbaI restriction sites of a pGS-Ex1 vector using the In-fusion HD cloning kit (#PT5162-1, a product of Clontech) to manufacture expression vectors of pEX1-1502, pEX1-1503, and pEX1-0776 for NCgl 1502, NCgl 1503, and NCgl 0776 genes, respectively. FIG. 5 is a cleavage map of the pEX1-1502 vector, FIG. 6 is a cleavage map of the pEX1-1503 vector, and FIG. 7 is a cleavage map of the pEX1-0776 vector.

pEX1-1502, pEX1-1503, and pEX1-0776 vectors were introduced into the D003 strain manufactured above by electroporation. Thereafter, the product was cultured in a LB culture medium, frozen, and stored.

### Example 5

### Evaluation of Productivity of 4-Hydroxybutyate

The control group strain D0003 (CGL (Δldh, ΔgD³, G3G) manufactured in Example 3) and the D003(+pEX1-1502), D003(+pEX1-1503), and D003(+pEX1-0776) strains manufactured in Example 4 were seed-cultured in 25 mL of brain heart infusion (BHI) culture medium (a product of Becton, Dickinson and Company) contained in a 125 mL flask at a temperature of 30° C., stirring at a rate of 230 rpm for 8 hours. The obtained culture was inoculated in 37.5 mL of a CgXII culture medium (Keilhauer et al. J. Bacteriol. 1993, 175:5595) contained in a 125 mL flask at a concentration of 1/50, and then cultured at a temperature of 30° C., stirring at a rate of 230 rpm for 16 hours. The flask was configured with a vented cap to allow an aerobic culture. Thereafter, cap and neck portions of the flask were sealed to create an anaerobic or microaerobic condition and the inoculated culture medium was cultured at a temperature of 30° C., stirring at a rate of 230 rpm for 24 hours. The final culture medium obtained therefrom was centrifuged, the supernatant obtained therefrom was filtered by using a syringe filter having a diameter of 0.45 um, and 4-hydroxybutyric acid (4HB) content was analyzed by using UPLC. The analysis was performed by using a conventional method as disclosed in J. Chromatogra. B, 885-886, 2012, 37-42. As an apparatus for the analysis, UPLC (a product of Waters) mounted with Aminex HPX-87H column (300 mm×7.8 mm, a product of Bio-Rad) was used. As a mobile phase, 2.5 mM sulfuric acid was used at a flow rate of 0.6 ml/min, and the column was maintained at a temperature of 60° C. Measurement of a refractive index and detection of a diode array were performed by using a detector.

Table 2 shows measurement results of the amount of 4HB produced by culturing the D003 (CGL (Δldh, ΔgD³, G3G)), D003(pEX1-1502), D003(pEX1-1503), and D003(pEX1-0776).

TABLE 2

| Strain | Amount of 4HB produced (mg/L) |
|---|---|
| D003 | 1110 |
| D003 (pEX1-1502) | 1373 |
| D003 (pEX1-1503) | 1297 |
| D003 (pEX1-0776) | 1367 |

As shown in Table 2, three strains in which the iron-regulated ABC transporter was over-expressed had increased 4HB production compared to the control group (strain D003) by 24%, 17%, and 23%, respectively.

Also, the method of producing hydroxycarboxylic acid has high efficiency.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism having an increased iron-regulated ABC transporter activity in comparison with a parent cell of the microorganism, and producing an increased amount of hydroxycarboxylic acid as compared to a parent cell of the microorganism.

2. The recombinant microorganism of claim 1, wherein the transporter is (a) SufB, (b) SufD, (c) ABC-type cobalamin/Fe³⁺-siderophore transport system, periplasmic component, or (d) a combination thereof.

3. The recombinant microorganism of claim 1, wherein
the SufB comprises an amino acid sequence having a sequence identity of 95% or more to SEQ ID NO: 1,
the SufD comprises an amino acid sequence having a sequence identity of 95% or more to SEQ ID NO: 3, and
the ABC-type cobalamin/Fe³⁺-siderophore transport system, periplasmic component comprises an amino acid sequence having a sequence identity of 95% or more to SEQ ID NO: 5.

4. The recombinant microorganism of claim 1, wherein the recombinant microorganism has a genetic modification that increases the activity of the transporter as compared to an activity of the parent cell of the microorganism.

5. The recombinant microorganism of claim 1, wherein the recombinant microorganism comprises an exogenous gene that encodes the transporter.

6. The recombinant microorganism of claim 5, wherein the exogenous gene encodes an amino acid sequence having a sequence identity of 95% or more to SEQ ID NOS: 1, 3, or 5.

7. The recombinant microorganism of claim 1, wherein the hydroxycarboxylic acid has a chemical structure represented by Formula 1 below:
HO—R¹—COOH [Formula 1]
wherein, R¹ is a straight or branched C₁-C₂₀ alkyl group.

8. The recombinant microorganism of claim 7, wherein, R¹ is a straight unsubstituted C₁-C₉ alkyl group.

9. The recombinant microorganism of claim 1, wherein the hydroxycarboxylic acid is 3-hydroxypropionic acid, 4-hydroxybutyric acid, 3-hydroxy-2-methylpropionic acid, 3-hydroxybutanoic acid, 3-hydroxy-2-methylbutanoic acid, 3-hydroxy-2-methylpentanoic acid, 3-hydroxy-3-methylbutanoic acid, 2,3-dimethyl-3-hydroxybutanoic acid, 3-hydroxy-3-phenylpropionic acid, or a combination thereof.

10. The recombinant microorganism of claim 1, wherein the recombinant microorganism belong to Escherichia sp, Rumen bacteria sp, Corynebacterium sp, or Brevibacterium sp.

11. The recombinant microorganism of claim 1, wherein the recombinant microorganism further comprises at least one of an exogenous gene encoding an enzyme that catalyzes conversion of succinic acid to succinyl-CoA, an exogenous gene encoding an enzyme that catalyzes conversion of succinyl-CoA to succinic semialdehyde (SSA), and an exogenous gene encoding an enzyme that catalyzes conversion of SSA to 4-hydroxybutyric acid.

12. The recombinant microorganism of claim 11, wherein the enzyme catalyzing conversion of succinic acid to succinyl-CoA is categorized as EC.2.8.3.-, the enzyme catalyzing conversion of succinyl-CoA to SSA is categorized as EC.1.2.1.16, and the enzyme catalyzing conversion of SSA to 4-hydroxybutyric acid is categorized as EC.1.1.1.1.

13. The recombinant microorganism of claim 1, wherein the enzyme catalyzing conversion of succinic acid to succinyl-CoA comprises an amino acid sequence having a sequence identity of 95% or more to SEQ ID NO: 7, 9, or 11; the enzyme catalyzing conversion of succinyl-CoA to SSA comprises an amino acid sequence having a sequence identity of 95% or more to SEQ ID NO: 13; and the enzyme catalyzing conversion of SSA to 4-hydroxybutyric acid comprises an amino acid sequence having a sequence identity of 95% or more to SEQ ID NO: 15.

14. The recombinant microorganism of claim 11, wherein the exogenous gene encoding the enzyme that catalyzes conversion of succinic acid to succinyl-CoA comprises a nucleotide sequence having a sequence identity of 95% or more to SEQ ID NO: 8, 10, or 12; the exogenous gene encoding the enzyme that catalyzes conversion of succinyl-CoA to SSA comprises a nucleotide sequence having a sequence identity of 95% or more to SEQ ID NO: 14; and the exogenous gene encoding the enzyme that catalyzes conversion of SSA to 4-hydroxybutyrat comprises a nucleotide sequence having a sequence identity of 95% or more to SEQ ID NO: 16.

15. The recombinant microorganism of claim 1, wherein the activity of at least one an enzyme catalyzing conversion of pyruvate into lactate or an enzyme catalyzing conversion of SSA into succinic acid is reduced in the recombinant microorganism as compared to a parent cell of the recombinant microorganism.

16. The recombinant microorganism of claim 15, wherein at least one of a gene encoding an enzyme that catalyzes conversion of pyruvate into lactate and a gene encoding an enzyme that catalyzes conversion of SSA into succinic acid is disrupted or deleted in the recombinant microorganism.

17. The recombinant microorganism of claim 15, wherein the enzyme catalyzing conversion of pyruvate into lactate is categorized as EC.1.1.1.27 and the enzyme catalyzing conversion of SSA into succinic acid is categorized as EC.1.2.1.16 or EC.1.2.1.39.

18. The recombinant microorganism of claim 17, wherein the enzyme catalyzing conversion of pyruvate into lactate comprises an amino acid sequence having a sequence identity of 95% or more to SEQ ID NO: 27 and the enzyme catalyzing conversion of SSA into succinic acid comprises an amino acid sequence having a sequence identity of 95% or more to SEQ ID NO: 21, 22, or 23.

19. The recombinant microorganism of claim 16, wherein the gene encoding the enzyme that catalyzes conversion of pyruvate into lactate comprises a nucleotide sequence having a sequence identity of 95% or more to SEQ ID NO: 89 and the gene encoding the enzyme that catalyzes conversion of SSA into succinic acid comprises a nucleotide sequence having a sequence identity of 95% or more to SEQ ID NO: 24, 25, or 26.

20. A method of producing hydroxycarboxylic acid, comprising:
culturing the recombinant microorganism of claim 1 with a carbon source; and
recovering hydroxycarboxylic acid from the culture.

21. The method of claim 20, wherein the culturing is performed in a microaerobic condition.

22. A method of producing a recombinant microorganism with improved hydroxycarboxylic acid production, the method comprising introducing an exogenous gene encoding an iron-regulated ABC transporter into a hydroxycarboxylic acid producing microorganism to provide a recombinant microorganism with improved hydroxycarboxylic acid production.


SEQUENCE LISTING (90 sequences)

SEQ ID NO: 1 - protein, 392 aa, Corynebacterium glutamicum
MTEVATVKAATPHNTKGDLFSSFNVEDFEIPRGRDEEWRFISLRRLRGLHNGEFAPATTSDVTVEIPADAEGASHEVVAKDDARLGRAGAPVDRVAAQAWTSKEQGNVVTFAKNSHNPTPVTITVTGKGEGVTSFGAISIEVEAGADAIVALQYVGSGTHADNVEFIVGDNARLTVITDTHWNADAVHLSNQLAQLGRDATLRHTVATFGGEVVRIVPRVRFTAPGGDAEMLGVYFADDGQYFEQRLLVDHAVPNCRSNVLYKGALQGDKNSDKPDARTCWVGDVLIRSNAHGTDTYEANRSLVLTEGARADAIPNLEIETGQIVGAGHAATVGRFDDEHVFYLQARGIPAEEARRLIVRGFFNEVINKVPVESIRGELDNRVSSELAVLGM

SEQ ID NO: 2 - DNA, 1179 bp, Corynebacterium glutamicum
atgactgaagtagcaaccgtaaaggccgcaaccccgcacaacaccaagggcgacctcttctcctcatttaatgttgaggactttgagatcccacgcggacgcgacgaagaatggcgtttcatttccctacgccgtcttcgcggactccacaacggcgagttcgcacctgcaaccacctctgacgtcaccgtcgagatccctgcagatgcagaaggcgcaagccacgaagttgtcgcaaaggacgatgctcgactgggtcgcgcaggcgcaccagtagaccgcgtcgcagctcaggcctggacctccaaggaacagggcaatgttgtcacctttgccaaaaactcccacaacccaaccccagtaaccatcacggttactggcaagggtgaaggcgttacctctttcggcgcaatctccatcgaggttgaagcgggcgcagacgctatcgtcgcactgcagtacgtcggatccggcacccacgctgacaacgtcgaattcatcgttggcgacaacgcacgcctgaccgtcatcacggacacccactggaacgctgacgcagttcacctgagcaaccagcttgcacagctgggacgcgacgcaactctacgccacaccgtggcaaccttcggtggagaagtagtccgcatcgtcccacgcgtgcgtttcaccgcaccaggtggcgacgcagaaatgctcggcgtctacttcgcagatgatggacagtacttcgagcagcgcctgctggttgaccacgctgtaccaaactgtcgctccaacgtcttgtacaagggcgcacttcagggtgacaagaactctgacaagccagatgcccgtacctgctgggttggcgatgtgctcatccgctcaaacgcccacggcactgacacctacgaagctaaccgctcactcgtcctcaccgagggtgcacgcgcagacgctattccaaacctcgagattgaaaccggccagatcgttggcgcaggacacgcagcaaccgtcggtcgtttcgacgacgagcacgtgttctacctccaggcccgtggtattcctgcagaggaagcacgccgcctcatcgtccgcggtttcttcaacgaagtgatcaacaaggtcccagttgaatccatccgcggggaattggacaaccgagtcagctcggaactcgcagttcttggcatgtaa

SEQ ID NO: 3 - protein, 481 aa, Corynebacterium glutamicum
MTSATTNPGVNEPLTDDQIIESIGPYNYGWHDSDDAGASAQRGLSEDVVRDISAKKSEPEWMLQQRLKALSIFDKKPVPTWGADLSGIDFDNIKYFVRSTEKQAQSWEDLPEDIKNTYDKLGIPEAEKQRLVAGVAAQYESEVVYHQIREDLEEKGVIFLDTDTALKEHPEIFQEYFGTVIPAGDNKFSALNSAVWSGGSFIYVPKGVHVDIPLQAYFRINTENMGQFERTLIIVDEDAYVHYVEGCTAPIYKSDSLHSAVVEIIVKKGGRCRYTTIQNWSNNVYNLVTKRTKVEEGGTMEWVDGNIGSKVTMKYPAVWMTGPHAKGEVLSVAFAGEGQFQDTGAKMTHMAPYTSSNIVSKSVARGGGRAAYRGLVQINANAHHSTSNVECDALLVDDISRSDTYPYNDIRNDHVSLGHEATVSQVSEEQLFYLMSRGLAEEEAMAMIVRGFVEPIAKELPMEYALELNRLIELQMEGSVG

SEQ ID NO: 4 - DNA, 1446 bp, Corynebacterium glutamicum
atgacttcggcaacgacgaacccaggggttaacgagcccttgaccgatgaccagatcattgaatccatcggtccgtacaactatggttggcacgactccgacgacgctggtgcatccgcacagcgtggtctcagcgaggatgtcgtacgcgacatctctgcgaagaagagcgagccagaatggatgcttcagcagcgcctcaaggccctgagcatttttgataagaagccagttccaacctggggtgcagacctttcaggcattgacttcgacaacattaaatacttcgtccgctccactgagaagcaggcacagtcctgggaggatctcccagaagacatcaagaatacctacgacaagctgggtattcctgaggccgagaagcagcgcctcgttgcaggtgttgcagctcagtacgagtctgaggttgtctaccaccagatccgcgaggacctggaggaaaagggagttatcttccttgacaccgataccgcactgaaagagcaccctgagatcttccaggagtacttcggcaccgtcattccagcaggcgacaacaagttctccgcactgaactccgctgtctggtccggtggatctttcatctacgtgccaaagggtgtccacgtggacattcctctgcaggcttacttccgcatcaacaccgagaacatgggtcagttcgaacgcaccctgatcatcgttgatgaggatgcctacgttcactacgttgagggctgtaccgcacctatttacaagtccgactccctgcactccgcagtcgttgagatcatcgtgaagaagggtggacgctgccgctacaccaccattcagaactggtccaacaacgtctacaacctggtgaccaagcgcaccaaggttgaagagggcggcaccatggaatgggtcgatggcaacatcggctccaaggtcaccatgaagtacccagctgtctggatgactggcccacacgcaaagggcgaagttctctccgtcgctttcgcaggtgagggacagttccaggacaccggcgccaagatgacccacatggctccttacacttcctccaacatcgtgtccaagtctgtggcacgtggcggtggacgtgcggcttaccgtggtctggttcagatcaacgcaaacgctcaccactcaacctccaacgttgagtgtgacgcactgctggtcgatgacatctcccgttctgacacctacccatacaacgacatccgtaacgatcacgtgtcactcggccacgaggcaactgtttcccaggtttctgaagagcagctgttctacctcatgagccgcggacttgcggaagaagaagcaatggcaatgatcgttcgtggcttcgttgagccaatcgctaaggaactcccaatggagtacgcccttgagctcaaccgactgatcgaactgcagatggaaggatcggtgggctaa

SEQ ID NO: 5 - protein, 338 aa, Corynebacterium glutamicum
MVKNRFKLVSIATVAALALVGCSSTDSTSSESSSAAESTAAASTLTIEDNHGTEGISLPIEGVAATDNRAFELLDRWGVELVAAPLQLVPFTVTGYTEEGGVANLGSHREPDLEALAAAQPSLIINGQRFAQYYDDIIALNPDATVVELDPRDGEPLDQELIRQAETLGEIFGEEEDAAKIVADFESALERAKTAYAAISDQTVMAVNVSGGNIGYIAPSVGRTYGPIFDLVGLTPALEVGNASSDHEGDDINVEAIAAANPDLILVMDRDGGTSTRNEADYVPAEQIVSDNEALANVKAVTDGYVYYAPADTYTNENIITYTEILNGMADMFEKAAQ

SEQ ID NO: 6 - DNA, 1017 bp, Corynebacterium glutamicum
atggtgaaaaaccgattcaagctagtttcaatcgcaactgttgctgccctggcgctcgttggctgctcttccaccgacagcacctcttccgagtcttcttccgctgcagagtcaaccgctgcagctagcaccctgactatcgaagacaaccacggcaccgaagggatctccctgccaatcgagggcgtcgctgcgaccgacaaccgcgcattcgaactgcttgatcgctggggtgtagagctcgttgcagctccacttcagctggttccatttaccgttacgggctacaccgaagagggcggcgtcgctaaccttggctcccaccgcgagccagacctggaagcacttgctgctgcacagccttccctgatcatcaacggccagcgcttcgctcagtactacgatgacatcattgccctgaaccctgacgcaaccgttgttgagctagacccacgcgatggcgagccacttgaccaggagcttatccgccaggctgaaaccctcggtgagatcttcggcgaagaagaagatgctgcaaagatcgttgctgatttcgagtccgcacttgagcgcgctaagaccgcatacgcagcaatctccgaccagaccgtcatggcagttaacgtttccggcggaaacattggctacatcgctccttccgttggacgcacctacggcccaatcttcgacctggttggactcaccccagcactcgaggttggcaacgcgtcctccgaccacgagggcgacgacattaacgtcgaagcaatcgcagctgcaaacccagacctgatcctggtcatggaccgcgatggtggcaccagcacccgcaacgaagctgattacgttccagcagagcagatcgtctccgacaatgaagcactggcaaacgtcaaggctgtcaccgacggatacgtttactacgcacctgcagatacctacaccaacgaaaacatcatcacctacaccgagatcctcaacggcatggcagatatgttcgagaaggcagctcagtag

SEQ ID NO: 7 - protein, 538 aa, Clostridium kluyveri
MSKGIKNSQLKKKNVKASNVAEKIEEKVEKTDKVVEKAAEVTEKRIRNLKLQEKVVTADVAADMIENGMIVAISGFTPSGYPKEVPKALTKKVNALEEEFKVTLYTGSSTGADIDGEWAKAGIIERRIPYQTNSDMRKKINDGSIKYADMHLSHMAQYINYSVIPKVDIAIIEAVAITEEGDIIPSTGIGNTATFVENADKVIVEINEAQPLELEGMADIYTLKNPPRREPIPIVNAGNRIGTTYVTCGSEKICAIVMTNTQDKTRPLTEVSPVSQAISDNLIGFLNKEVEEGKLPKNLLPIQSGVGSVANAVLAGLCESNFKNLSCYTEVIQDSMLKLIKCGKADVVSGTSISPSPEMLPEFIKDINFFREKIVLRPQEISNNPEIARRIGVISINTALEVDIYGNVNSTHVMGSKMMNGIGGSGDFARNAYLTIFTTESIAKKGDISSIVPMVSHVDHTEHDVMVIVTEQGVADLRGLSPREKAVAIIENCVHPDYKDMLMEYFEEACKSSGGNTPHNLEKALSWHTKFIKTGSMK

SEQ ID NO: 8 - DNA, 1617 bp, Clostridium kluyveri
atgtctaaaggaatcaagaatagccaattgaaaaaaaagaacgtcaaggccagtaacgttgctgagaagatcgaagagaaggtggaaaagaccgacaaggtcgttgagaaggctgctgaggtgaccgaaaagcgcattcgaaacttaaagctccaggaaaaagttgtgaccgcagatgtcgcagctgacatgatcgagaatggcatgatcgtcgcaattagcggcttcacgccatccgggtatccaaaggaggttccaaaagcccttactaagaaggttaatgcgctggaggaggagttcaaggtgacgctgtataccggttctagcacaggcgctgatattgacggagaatgggcgaaggcaggaataatcgaacggcgtatcccataccagaccaactctgacatgaggaaaaaaataaacgatggttcaatcaagtacgcagatatgcacctgagccacatggctcaatacattaactattctgtgattcctaaggttgacattgccatcatcgaggcggtggccattaccgaggaaggggatattattcctagtactggaatcggcaacacagctacgtttgtcgagaatgcggataaggtaattgtggaaataaacgaggctcagccgcttgagttggaaggcatggcagatatctataccctgaagaaccctccacgtcgcgagcccatcccgatagtcaacgcaggcaaccgcatagggaccacttacgtcacctgtggctctgaaaaaatctgcgcgatcgtcatgaccaacacccaagacaaaacccgcccactcaccgaagtttctcctgtcagtcaggcaatctccgataacctgattggcttcctgaacaaagaagtagaggagggtaaactcccaaaaaacctgctccccatacagtcaggtgtcggttcggttgctaacgccgttctagccggactctgcgaatcaaacttcaaaaatttgagctgctacacagaagtgatccaggattcgatgttgaagctcatcaaatgtggaaaggcagatgtggtgtccggcacctcgatctcgccatcaccggaaatgctgcccgagttcataaaggacataaatttttttcgcgagaagatagtactgcgcccccaggaaatatctaataatccggaaatagctcgtcgtataggagtgatctccataaacactgctttggaagtagacatctacggtaatgtgaactccacgcatgtcatgggctccaagatgatgaacggcatcggcggcagcggcgactttgcccgcaacgcatacctcaccatattcactacggagtccatcgcgaagaagggcgacatttcctctatcgttcctatggtttcccacgtggaccacaccgagcatgacgtaatggtcatcgttaccgaacagggggttgcggatctgcgcggtctttcccctcgggaaaaggccgtggcgataattgagaattgcgtccacccggattacaaggatatgctcatggagtacttcgaggaggcttgtaagtcctcaggtggcaacaccccacacaaccttgaaaaagccctatcctggcacactaagttcataaaaactggctcgatgaagtaa

SEQ ID NO: 9 - protein, 502 aa, Corynebacterium glutamicum
MSDRIASEKLRSKLMSADEAAQFVNHGDKVGFSGFTGAGYPKALPTAIANRAKEAHGAGNDYAIDLFTGASTAPDCDGVLAEADAIRWRMPYASDPIMRNKINSGSMGYSDIHLSHSGQQVEEGFFGQLNVAVIEITRITEEGYIIPSSSVGNNVEWLNAAEKVILEVNSWQSEDLEGMHDIWSVPALPNRIAVPINKPGDRIGKTYIEFDTDKVVAVVETNTADRNAPFKPVDDISKKIAGNFLDFLESEVAAGRLSYDGYIMQSGVGNVPNAVMAGLLESKFENIQAYTEVIQDGMVDLIDAGKMTVASATSFSLSPEYAEKMNNEAKRYRESIILRPQQISNHPEVIRRVGLIATNGLIEADIYGNVNSTNVSGSRVMNGIGGSGDFTRNGYISSFITPSEAKGGAISAIVPFASHIDHTEHDVMVVISEYGYADLRGLAPRERVAKMIGLAHPDYRPLLEEYYARATSGDNKYMQTPHDLATAFDFHINLAKNGSMKA

SEQ ID NO: 10 - DNA, 1509 bp, Corynebacterium glutamicum
atgtctgatcgcattgcttcagaaaagctgcgctccaagctcatgtccgccgacgaggcggcacagtttgttaaccacggtgacaaggttggtttctccggcttcaccggcgctggctacccaaaggcactgcctacggcaatcgctaaccgggctaaagaagcacacggtgcaggcaacgactacgcaatcgacctgttcactggcgcatcgaccgcccctgactgcgatggcgtacttgcagaagctgacgctatccgctggcgcatgccatacgcatctgatccaatcatgcgtaacaagatcaactccggctccatgggatactccgatatccacctgtcccactccggccagcaggttgaagagggcttcttcggccagctcaacgtagctgtcattgaaatcacccgcatcactgaagagggctacatcatcccttcttcctccgtgggtaacaacgttgagtggctcaacgctgcagagaaggtcatcctcgaggttaactcttggcagtctgaagacctcgaaggtatgcacgacatctggtctgttcctgccctgccaaaccgcattgccgtgccaatcaacaagccaggcgaccgcatcggtaagacctacatcgagttcgacaccgacaaggttgttgctgttgttgagaccaacaccgcagaccgcaacgcaccattcaagcctgtcgacgacatctctaagaagatcgctggcaacttcctcgacttcctggaaagcgaagttgctgcaggtcgcctgtcctacgacggctacatcatgcagtccggcgtgggcaacgtgccaaacgcggtgatggcaggcctgctggaatccaagtttgagaacatccaggcctacaccgaagttatccaggacggcatggtggacctcatcgacgccggcaagatgaccgttgcatccgcaacttccttctccctgtctcctgagtacgcagagaagatgaacaacgaggctaagcgttaccgcgagtccattatcctgcgcccacagcagatctctaaccacccagaggtcatccgccgcgttggcctgatcgccaccaacggtctcatcgaggctgacatttacggcaacgtcaactccaccaacgtttctggctcccgcgtcatgaacggcatcggcggctccggcgacttcacccgtaacggctacatctccagcttcatcaccccttcagaggcaaagggcggcgcaatctctgcgatcgttcctttcgcatcccacatcgaccacaccgagcacgatgtcatggttgttatctctgagtacggttacgcagaccttcgtggtctggctccacgtgagcgcgttgccaagatgatcggcctggctcaccctgattaccgcccactgctcgaggagtactacgctcgcgcaacctccggtgacaacaagtacatgcagacccctcatgatcttgcaaccgcgtttgatttccacatcaacctggctaagaacggctccatgaaggcataa

SEQ ID NO: 11 - protein, 125 aa, Corynebacterium glutamicum
MNGIGGSGDFTRNAFASTFISPSAAKVDAISAIVPFASHIDHTEHDAMVVITEYGYADLRGLSPKQRVPKMIAIAHPDYRPLLEAYFDRALNSADSYQHTLHDLRTAFDFHNRLNSQGTMKIEKA

SEQ ID NO: 12 - DNA, 378 bp, Corynebacterium glutamicum
atgaatggtatcggcggctcgggcgatttcacgcgtaacgcctttgcttccacatttatctctccctcggcagccaaagttgatgcgatttccgcgattgtgcctttcgcgtcccatatcgatcacacggaacatgatgcgatggttgtcattactgaatatggctacgcagacctgcgcgggctatcgccaaaacaacgagtccccaaaatgattgccatcgcccacccggactatcgaccactgctggaagcatactttgaccgggcgctgaacagtgctgattcctatcagcacaccctgcatgatctgcgcaccgccttcgatttccataatcgcttgaactcacaaggaaccatgaaaatcgaaaaagcatag

SEQ ID NO: 13 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 14 - DNA, 1356 bp, Porphyromonas gingivalis
atggagattaaagagatggtcagtcttgcgcgcaaagctcagaaggagtatcaggccacccataaccaagaagctgtggacaacatctgccgagcagcagcgaaggttatttacgaaaatgcagcaattctggcacgcgaggcagtggacgaaaccggcatgggtgtttacgagcacaaggtggccaagaatcaaggcaagtccaaaggtgtttggtacaacctgcataacaagaagtcgattggcatcctcaatatcgacgagcgtaccggcatgatcgagatcgcaaaacctatcggggttgtaggcgccgttacgccaaccaccaaccctatcgttactccgatgagcaacatcatctttgctcttaagacctgcaacgccatcattatcgccccacacccgcgctccaaaaagtgctctgcccacgcagttcggctgatcaaagaggctatcgctccgttcaacgtgcccgaaggtatggttcagatcatcgaggagcctagcatcgagaagacgcaggaattgatgggcgccgtagacgtggtcgttgctaccgggggcatgggcatggtcaagtctgcctactcctcagggaagccttctttcggtgtcggagccggcaatgttcaggtgatagtggacagcaacatcgacttcgaagcggcagcagaaaagatcatcaccggacgtgccttcgacaacggtatcatctgctcaggcgaacagtccatcatctacaacgaggctgacaaggaagcagttttcacagcattccgcaaccacggtgcgtacttttgcgacgaggccgagggagatcgggctcgtgcagcgatcttcgaaaatggagccatcgcgaaagatgttgtgggccagtccgttgcctttattgcaaagaaggcgaacattaatatccccgagggtactcgtattctcgtggtcgaagctcgcggagtaggcgccgaagatgtcatctgtaaagaaaagatgtgtccagtcatgtgcgccctctcctacaagcacttcgaagagggggtagagatcgcaaggacgaacctcgcaaacgaaggcaatggccatacctgtgctatccactccaacaaccaagcacacatcatcttggcaggctcggagctgaccgtgtctcgcatcgtggtcaacgcgccaagtgctaccacagcaggcggtcacatccagaacggtcttgccgtcaccaatactctaggctgcggctcttggggtaacaactcgatctccgaaaacttcacttataaacacctgctcaacatttcacgcatcgccccgttgaactccagcattcatatcccagatgataaggaaatctgggaactctaa

SEQ ID NO: 15 - protein, 371 aa, Porphyromonas gingivalis
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 16 - DNA, 1116 bp, Porphyromonas gingivalis
atgcagcttttcaagctcaagagcgtcacacatcactttgatacttttgcagagtttgccaaggagttctgtctcggtgaacgcgacttggtaattaccaacgagttcatctacgaaccgtatatgaaggcatgccagctgccttgtcattttgtgatgcaggagaaatacggccaaggcgagccttctgacgagatgatgaacaacatcctagcagatatccgtaatatccagttcgaccgcgtgatcgggatcggaggtggtacggttattgacatctcaaaactctttgttctgaagggattaaatgatgttctcgacgcgttcgatcgcaagattccccttatcaaagagaaagaactgatcattgtgcccaccacctgcggaaccggctcggaggtgacgaacatttccatcgccgagatcaagtcccggcacaccaagatgggtttggctgacgatgcaattgttgctgaccacgccataatcatccctgaacttctgaagagcttgcccttccacttctatgcatgctccgcaatcgacgctcttattcatgccatcgagtcatacgtttctccaaaagcgtctccatactcccgtctgttcagtgaggcggcgtgggacattatcctggaagttttcaagaaaatcgccgaacacggcccagagtaccgcttcgagaagctgggggaaatgatcatggccagcaactatgccggtatcgctttcggcaacgcaggcgttggcgccgtccacgctctatcctacccgttgggcggcaactatcacgtgccgcatggagaagcaaactatcagttcttcaccgaggtctttaaagtataccaaaagaagaatccgttcggctatattgtcgaactcaactggaagctctccaagattctgaactgccagccagagtacgtgtacccgaagctggatgaactgctcggttgccttcttaccaagaaacctttgcacgaatacggcatgaaggacgaagaggttcgtggcttcgcggaatcggtcctgaagacccagcaacgcttgctcgccaacaactacgtcgaacttactgtcgatgagatcgaaggtatctaccgacgtctctactag

SEQ ID NO: 17 - protein, 543 aa, Corynebacterium glutamicum
MSSTPAQDLARAVIDSLAPHVTDVVLCPGSRNSPLSLELLARQDLRVHVRIDERSASFLALSLARTQARPVAVVMTSGTAVANCLPAVAEAAHAHIPLIVLSADRPAHLVGTGASQTINQTGIFGDLAPTVGITELDQVAQIAESLAQGASQIPRHFNLALDVPLVAPELPELHGEAVGASWTHRWINHGEVTVDLGEHTLVIAGDEAWEVEGLEDVPTIAEPTAPKPYNPVHPLAAEILLKEQVSAEGYVVNTRPDHVIVVGHPTLHRGVLKLMSDPGIKLTVLSRTDIITDPGRHADQVGSTVKVTGTQEKQWLKICSAASELAADGVRDVLDNQEFGFTGLHVAAAVADTLGTGDTLFAAASNSIRDLSLVGMPFDGVDTFSPRGVAGIDGSVAQAIGTSLAVQSRHPDEIRAPRTVALLGDLSFLHDIGGLLIGPDEPRPENLTIVVSNDNGGGIFELLETGADGLRPNFERAFGTPHDASIADLCAGYGIEHQVVDNLQDLIIALVDTTEVSGFTIIEASTVRDTRRAQQQALMDTVH

SEQ ID NO: 18 - protein, 933 aa, Escherichia coli
MQNSALKAWLDSSYLSGANQSWIEQLYEDFLTDPDSVDANWRSTFQQLPGTGVKPDQFHSQTREYFRRLAKDASRYSSTISDPDTNVKQVKVLQLINAYRFRGHQHANLDPLGLWQQDKVADLDPSFHDLTEADFQETFNVGSFASGKETMKLGELLEALKQTYCGPIGAEYMHITSTEEKRWIQQRIESGRATFNSEEKKRFLSELTAAEGLERYLGAKFPGAKRFSLEGGDALIPMLKEMIRHAGNSGTREVVLGMAHRGRLNVLVNVLGKKPQDLFDEFAGKHKEHLGTGDVKYHMGFSSDFQTDGGLVHLALAFNPSHLEIVSPVVIGSVRARLDRLDEPSSNKVLPITIHGDAAVTGQGVVQETLNMSKARGYEVGGTVRIVINNQVGFTTSNPLDARSTPYCTDIGKMVQAPIFHVNADDPEAVAFVTRLALDFRNTFKRDVFIDLVCYRRHGHNEADEPSATQPLMYQKIKKHPTPRKIYADKLEQEKVATLEDATEMVNLYRDALDAGDCVVAEWRPMNMHSFTWSPYLNHEWDEEYPNKVEMKRLQELAKRISTVPEAVEMQSRVAKIYGDRQAMAAGEKLFDWGGAENLAYATLVDEGIPVRLSGEDSGRGTFFHRHAVIHNQSNGSTYTPLQHIHNGQGAFRVWDSVLSEEAVLAFEYGYATAEPRTLTIWEAQFGDFANGAQVVIDQFISSGEQKWGRMCGLVMLLPHGYEGQGPEHSSARLERYLQLCAEQNMQVCVPSTPAQVYHMLRRQALRGMRRPLVVMSPKSLLRHPLAVSSLEELANGTFLPAIGEIDELDPKGVKRVVMCSGKVYYDLLEQRRKNNQHDVAIVRIEQLYPFPHKAMQEVLQQFAHVKDFVWCQEEPLNQGAWYCSQHHFREVIPFGASLRYAGRPASASPAVGYMSVHQKQQQDLVNDALNVE

SEQ ID NO: 19 - DNA, 1632 bp, Corynebacterium glutamicum
atgtccagcacgccagctcaagatcttgcccgcgccgttattgattccctcgcaccacacgtcactgacgtggtgttatgcccaggatccaggaactcaccgttgtcgcttgagttgctggcgcggcaggatctgcgtgtccatgtgcgtatcgacgagcgcagcgcctcatttttggcgctgtccctagcgcgtacccaggcccggccggtggctgtggtgatgacctccggcacggctgtagctaactgcctgcctgctgttgctgaagctgcgcatgcccatatcccgttgattgtgctctctgctgaccgtcctgcacatttggtgggaacgggggcgagccaaacgattaaccagaccggtatttttggtgatcttgcaccgacggtcggtatcactgagctggatcaggtagcgcagattgctgaaagccttgctcagggggcttcccagattccgcgtcatttcaatcttgcacttgatgttcctttggttgctcctgaactgccagagcttcatggtgaggcagttggagcatcatggacgcatcgctggatcaaccacggtgaggtgaccgtggacctgggggagcacaccctcgtgattgccggtgatgaagcatgggaagtggaagggctggaagatgtgcccaccatcgctgaacctactgcaccaaagccttataatccggtgcacccactggctgctgaaatcttgctgaaggagcaggtctccgcggaaggctatgtggtaaacaccaggcctgatcatgtgatcgtggtgggacaccccacgctgcaccgcggagtgttgaagttgatgtcagatcctggcattaaattaactgtgctttcacgcaccgatatcatcactgatcccggccgccatgccgatcaggtgggcagcacagtgaaagtcaccggcacccaggaaaagcagtggctaaagatctgttcggcagcatcagaacttgcggccgatggtgtgcgtgacgtcctggacaaccaagaattcggtttcaccggcctccatgttgccgcagccgtggcggataccttaggcaccggcgatactctctttgctgcagcatccaactcaatccgtgacctctccctggtgggtatgccttttgatggcgtggataccttctccccacgaggtgtcgcaggcattgatggttctgttgctcaagcaatcggcacttcacttgctgtgcagtcccgccaccccgatgaaatccgcgcgccacgcactgtggcccttctgggcgatctgtcgttccttcacgatattggcggactgctcatcggccctgatgaaccacgcccagaaaacctcaccatcgtggtctccaacgacaacggtggcggaatcttcgaactcctagaaaccggcgcagatggtctccgccccaacttcgagcgtgctttcggtaccccacacgacgcgtccatcgcggatctctgcgcaggctacggcattgaacaccaagtggtagacaacctccaagacctcatcatcgcgctagttgataccaccgaagtatccggattcaccattattgaagcttcgaccgtccgagatacccgccgtgcacaacagcaagctctcatggacacggtgcactaa

SEQ ID NO: 20 - DNA, 2802 bp, Escherichia coli
atgcagaacagcgctttgaaagcctggttggactcttcttacctctctggcgcaaaccagagctggatagaacagctctatgaagacttcttaaccgatcctgactcggttgacgctaactggcgttcgacgttccagcagttacctggtacgggagtcaaaccggatcaattccactctcaaacgcgtgaatatttccgccgcctggcgaaagacgcttcacgttactcttcaacgatctccgaccctgacaccaatgtgaagcaggttaaagtcctgcagctcattaacgcataccgcttccgtggtcaccagcatgcgaatctcgatccgctgggactgtggcagcaagataaagtggccgatctggatccgtctttccacgatctgaccgaagcagacttccaggagaccttcaacgtcggttcatttgccagcggcaaagaaaccatgaaactcggcgaactgctggaagcactcaagcaaacctactgcggcccgattggtgccgagtatatgcacattaccagcactgaagaaaaacgctggatccaacagcgtattgagtctggtcgcgcgactttcaatagcgaagagaaaaaacgcttcttaagcgaactgaccgccgctgaaggccttgaacgttacctcggcgcaaaattccctggcgcaaaacgcttctcgctggaaggcggtgacgcgttaatcccgatgcttaaagagatgatccgccacgctggcaacagcggcacccgcgaagtggttctcggaatggcgcaccgtggtcgtctgaacgtgctggtgaacgtgctgggtaaaaaaccgcaagacttgttcgacgagtttgccggtaaacataaagaacacctcggcacgggcgacgtgaaataccacatgggcttctcgtctgacttccagaccgatggcggcctggttcacctggcgctggcgtttaacccgtctcaccttgagattgtaagcccggtcgttatcggttctgttcgtgcccgtctggacagacttgatgagccgagcagcaacaaagtgctgccaatcaccattcatggtgacgccgcagtgaccgggcagggcgtggttcaggaaaccctgaacatgtcgaaagcgcgtggttatgaagttggcggtacggtacgtatcgttatcaacaaccaggttggcttcaccacctctaacccgctggatgcccgttcgacgccgtactgtactgatatcggtaagatggttcaggcaccgattttccacgttaacgcggatgatccggaagccgttgcctttgttacccgtctggcgctcgatttccgtaacacctttaaacgtgatgtcttcatcgacctggtatgctaccgccgtcacggccacaacgaagccgacgagccgagcgcaacccagccgctgatgtatcagaaaatcaaaaaacatccgacgccgcgcaaaatctacgctgacaagctggagcaggaaaaagtcgcgacgctggaagatgccaccgagatggttaacctgtaccgcgatgcgctggatgctggcgattgcgttgtagcagagtggcgtccgatgaacatgcactctttcacctggtcgccgtacctcaaccatgaatgggacgaagagtacccgaacaaagttgagatgaagcgcctgcaggaactggctaaacgcatcagcacggtgccggaagcggttgaaatgcagtctcgcgttgccaagatttatggcgatcgccaggcgatggcagccggtgagaaactgttcgactggggcggcgcggaaaacctcgcttacgccacgttggttgacgaaggcattccggttcgcctgtcgggtgaagactccggtcgcggtaccttcttccaccgccacgcggtgatccacaaccagtctaacggttccacttacacgccgctgcaacatatccataacggccagggcgcgttccgcgtctgggactctgtactgtctgaagaagccgtactggcgtttgaatacggttatgccaccgcagaaccacgcaccctgaccatctgggaagcacagttcggtgacttcgccaacggtgcacaggtggttatcgaccagttcatctcctctggcgaacagaaatggggccggatgtgtggcctggtgatgttgctgccgcacggttacgaagggcaggggccggagcactcctccgcgcgtctggaacgttatctgcaactttgcgctgagcaaaacatgcaggtttgcgtaccttctaccccggcacaggtttaccacatgctgcgtcgtcaggcgttgcgcgggatgcgtcgtccactggtcgtgatgtcgccgaaatccctgctgcgtcatccgctggcggtatccagcctcgaagaactggcgaacggcaccttcctgccagccatcggtgaaatcgacgagcttgatccgaagggcgtgaagcgcgtagtgatgtgttctggtaaggtttattacgacctgctggaacaacgtcgtaagaacaatcaacacgatgtcgccattgtgcgtatcgagcaactctacccgttcccgcataaagcgatgcaggaagtgttgcagcagtttgctcacgtcaaggattttgtctggtgccaggaagagccgctcaaccagggcgcatggtactgcagccagcatcatttccgtgaagtgattccgtttggggcttctctgcgttatgcaggccgcccagcctccgcctctccggcggtagggtatatgtccgttcaccagaaacagcaacaagatctggttaatgacgcgctgaacgtcgaataa

SEQ ID NO: 21 - protein, 490 aa, Corynebacterium glutamicum
MTINVSELLAKVPTGLLIGDSWVEASDGGTFDVENPATGETIATLASATSEDALAALDAACAVQAEWARMPARERSNILRRGFELVAERAEEFATLMTLEMGKPLAEARGEVTYGNEFLRWFSEEAVRLYGRYGTTPEGNLRMLTALKPVGPCLLITPWNFPLAMATRKVAPAIAAGCVMVLKPARLTPLTSQYFAQTMLDAGLPAGVLNVVSGASASAISNPIMEDDRLRKVSFTGSTPVGQQLLKKAADKVLRTSMELGGNAPFIVFEDADLDLAIEGAMGAKMRNIGEACTAANRFLVHESVADEFGRRFAARLEEQVLGNGLDEGVTVGPLVEEKARDSVASLVDAAVAEGATVLTGGKAGTGAGYFYEPTVLTGVSTDAAILNEEIFGPVAPIVTFQTEEEALRLANSTEYGLASYVFTQDTSRIFRVSDGLEFGLVGVNSGVISNAAAPFGGVKQSGMGREGGLEGIEEYTSVQYIGIRDPYAG

SEQ ID NO: 22 - protein, 453 aa, Corynebacterium glutamicum
MSLTFPVINPSDGSTITELENHDSTQWMSALSDAVAAGPSWAAKTPRERSVVLTAIFEALTERAQELAEIIHLEAGKSVAEALGEVAYGAEYFRWFAEEAVRLPGRYGQSPSGIGHIAVTRAPVGPVLAITPWNFPIAMATRKIAPALAAGCPVLVKPASETPLTMVKVGEIIASVFDTFNIPQGLVSIITTTRDAELSAELMADPRLAKVTFTGSTNVGRILVRQSADRLLRTSMELGGNAAFVIDEAADLDEAVSGAIAAKLRNAGQVCIAANRFLVHESRAAEFTSKLATAMQNTPIGPVISARQRDRIAALVDEAITDGARLIIGGEVPDGSGFFYPATILADVPAQSRIVHEEIFGPVATIATFTDLAEGVAQANSTEFGLAAYGFSNNVKATQYMAEHLEAGMVGINRGAISDPAAPFGGIGQSGFGREGGTEGIEEYLSVRYLALP

SEQ ID NO: 23 - protein, 521 aa, Corynebacterium glutamicum
MIKRLPLGPLPKELHQTLLDLTANAQDAAKVEVIAPFTGETLGFVFDGDEQDVEHAFALSRAAQKKWVHTTAVERKKIFLKFHDLVLKNRELLMDIVQLETGKNRASAADEVLDVAITTRFYANNAGKFLNDKKRPGALPIITKNTQQYVPKGVVGQITPWNYPLTLGVSDAVPALLAGNAVVAKPDLATPFSCLIMVHLLIEAGLPRDLMQVVTGPGDIVGGAIAAQCDFLMFTGSTATGRILGRTMGERLVGFSAELGGKNPLIVAKDADLDKVEAELPQACFSNSGQLCVSTERIYVEEDVYEEVIARFSKAAKAMSIGAGFEWKYEMGSLINQAQLDRVSTFVDQAKAAGATVLCGGKSRPDIGPFFYEPTVLADVPEGTPLLTEEVFGPVVFIEKVATLEEAVDKANGTPYGLNASVFGSSETGNLVAGQLEAGGIGINDGYAATWASVSTPLGGMKQSGLGHRHGAEGITKYAEIRNIAEQRWMSMRGPAKMPRKVYSDTVATALKLGKIFKVLP

SEQ ID NO: 24 - DNA, 1473 bp, Corynebacterium glutamicum
atgactattaatgtctccgaactacttgccaaagtccccacgggtctactgattggtgattcctgggtggaagcatccgacggcggtactttcgatgtggaaaacccagcgacgggtgaaacaatcgcaacgctcgcgtctgctacttccgaggatgcactggctgctcttgatgctgcatgcgctgttcaggccgagtgggctaggatgccagcgcgcgagcgttctaatattttacgccgcggttttgagctcgtagcagaacgtgcagaagagttcgccaccctcatgaccttggaaatgggcaagcctttggctgaagctcgcggcgaagtcacctacggcaacgaattcctgcgctggttctctgaggaagcagttcgtctgtatggccgttacggaaccacaccagaaggcaacttgcggatgctgaccgccctcaagccagttggcccgtgcctcctgatcaccccatggaacttcccactagcaatggctacccgcaaggtcgcacctgcgatcgctgcaggttgtgtcatggtgctcaagccagctcgacttaccccgctgacctcccagtattttgctcagaccatgcttgatgccggtcttccagcaggtgtcctcaatgtggtctccggtgcttccgcctctgcgatttccaacccgattatggaagacgatcgccttcgtaaagtctccttcaccggctccaccccagttggccagcagctgctcaaaaaggctgccgataaagttctgcgcacctccatggaacttggtggcaacgcacctttcattgtcttcgaggacgccgacctagatctcgcgatcgaaggtgccatgggtgccaaaatgcgcaacatcggcgaagcttgcaccgcagccaaccgtttcttagtccacgaatccgtcgccgatgaattcggccgtcgcttcgctgcccgccttgaagagcaagtcctaggcaacggcctcgacgaaggcgtcaccgtgggccccctggttgaggaaaaagcacgagacagcgttgcatcgcttgtcgacgccgccgtcgccgaaggtgccaccgtcctcaccggcggcaaggccggcacaggtgcaggctacttctacgaaccaacggtgctcacgggagtttcaacagatgcggctatcctgaacgaagagatcttcggtcccgtcgcaccgatcgtcaccttccaaaccgaggaagaagccctgcgtctagccaactccaccgaatacggactggcctcctatgtgttcacccaggacacctcacgtattttccgcgtctccgatggtctcgagttcggcctagtgggcgtcaattccggtgtcatctctaacgctgctgcaccttttggtggcgtaaaacaatccggaatgggccgcgaaggtggtctcgaaggaatcgaggagtacacctccgtgcagtacatcggtatccgggatccttacgccggctag

SEQ ID NO: 25 - DNA, 1362 bp, Corynebacterium glutamicum
gtgtctttgaccttcccagtaatcaaccccagcgatggctccaccatcaccgagctagaaaaccacgattccacccagtggatgtccgcgctctctgatgcagttgcagctggtccttcatgggctgcgaaaactccccgcgaaagatccgtggtactcaccgcaatcttcgaagcactgaccgaacgcgcccaagaacttgcagagatcatccacctggaagctggaaaatccgttgcagaagctcttggtgaagtcgcttatggtgcagaatacttccgttggtttgcggaagaagcagtgcgcctgcccggccgctacggacagtcaccttccggaatcggtcacatcgccgtcacccgcgcacccgtgggaccagtgctggcgatcaccccatggaatttccccatcgccatggccacccgcaaaatcgccccagccctggccgctggttgccccgtgttggtgaaacctgcttccgaaaccccactgaccatggtcaaagtgggggagatcatcgcctccgtctttgatacctttaatatcccgcagggcttggtctcaatcatcaccaccactcgagatgcagagctatcggcagaactcatggctgatcctcgcttggctaaagtcaccttcactggatcaaccaacgtgggacgcatcctggtccgccaatccgcggaccgactgctgcgcacctccatggaactcggcggaaatgcagcttttgttatcgacgaagccgcagacctcgacgaagccgtatccggtgccatcgccgcaaaactccgcaacgccggccaagtatgcatcgcagctaaccgtttcttggttcatgaatcccgcgctgccgaattcacctcaaagctggcgacagccatgcagaacactcccattgggccggtgatttctgcccgccaacgcgaccggatcgcagcactagtggatgaagccatcaccgacggcgcccgcctcatcatcggtggggaggtccccgacggctccggcttcttctatccagccaccatcttggccgatgtccctgcacagtcacggattgtgcatgaggaaatcttcggacctgtggccaccattgccactttcaccgacttggccgaaggcgttgcacaagcaaattccaccgaattcggcctcgcagcctacggattcagcaacaatgtgaaagcaacacagtacatggcggaacacttggaagccggaatggtcggaatcaacagaggcgccatctctgacccagcagcaccttttggcggcatcggacaatccggcttcggcagagaaggcggaaccgaaggaatcgaagaatatctctccgtgcgttacctcgctttgccgtga

SEQ ID NO: 26 - DNA, 1566 bp, Corynebacterium glutamicum
atgatcaaacgtcttcctttaggtccgctgcctaaagaacttcatcagactctgcttgatctgaccgcaaatgcccaagatgcggcgaaagtggaggttatagcgccatttactggcgagaccctcggatttgtttttgatggtgatgagcaagacgtcgagcatgcttttgcactttcaagggcagcccagaaaaagtgggtgcacaccacggcagtggaacggaagaagatcttcctgaagtttcatgatctggtattgaaaaaccgtgagctgctcatggacatcgtgcagttggaaacaggcaaaaatcgagcatcggctgccgatgaggtgttggacgttgcgatcaccacccgcttctacgcaaacaatgcaggaaagtttttaaatgacaagaaacgccccggcgcgcttccgatcatcacgaaaaacacacaacagtatgtgcccaagggagtggtcgggcagatcacgccgtggaattaccctttaactttgggagtatctgatgctgttccggcgctgctggcaggaaacgcagtggtggctaaacctgacctcgcgacacctttctcctgcttgatcatggtgcacctgctcattgaagccggtctgccgcgtgatttgatgcaggttgtcaccggccctggcgatattgttggcggtgcgattgcagctcagtgtgatttcctcatgttcactggatccacggccacgggccggatcttgggtcggacaatgggtgagcgtttggtgggtttctctgcggaattaggcggaaagaaccctcttattgtggccaaggatgcagatctggacaaggtggaagctgagcttccgcaggcgtgtttttccaactcggggcaattgtgtgtctccactgaacgtatttatgtcgaggaagacgtgtacgaggaggtgattgcacggtttagcaaggcggcgaaagccatgtccattggtgccggatttgagtggaaatatgagatgggttcgttgatcaatcaggcgcagctggatcgggtgagcacctttgttgatcaggctaaagctgcgggcgccacggtgctgtgcggtggcaagtcacgccctgatattggtcccttcttctatgagcccacggtattggcggatgtcccagagggcaccccactgctcacggaggaagtcttcgggccggtggtgttcatcgaaaaggtagccacactggaagaagccgtcgataaggcaaatggcacgccctacggcctgaatgcgtccgtctttgggtcgtcggaaaccggcaatcttgttgcaggccagctggaagctggcggtatcggtattaatgatggctacgccgcgacgtgggcgagcgtgtccacgcctctgggtggcatgaagcagtcggggctggggcaccgccatggtgcggagggaattacaaaatatgcggagatccgaaacatcgcggagcagcgctggatgtctatgcgtgggccggccaaaatgccgcgaaaggtgtactcagacaccgtggccacagcgctaaagctgggcaaaatctttaaagttttgccgtag

SEQ ID NO: 27 - protein, 314 aa, Corynebacterium glutamicum
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 28 - DNA, 43 bp, Artificial Sequence
  Synthetic (ldhA_5′_HindIII)
catgattacgccaagcttgagagcccaccacattgcgatttcc

SEQ ID NO: 29 - DNA, 42 bp, Artificial Sequence
  Synthetic (ldhA_up_3′_XhoI)
tcgaaactcgagtttcgatcccacttcctgatttccctaacc

SEQ ID NO: 30 - DNA, 39 bp, Artificial Sequence
  Synthetic (ldhA_dn_5′_XhoI)
tcgaaactcgagtaaatctttggcgcctagttggcgacg

SEQ ID NO: 31 - DNA, 46 bp, Artificial Sequence
  Synthetic (ldhA_3′_EcoRI)
acgacggccagtgaattcgacgacatctgagggtggataaagtggg

SEQ ID NO: 32 - DNA, 20 bp, Artificial Sequence
  Synthetic (ldhA up)
atcgggcataattaaaggtg

SEQ ID NO: 33 - DNA, 22 bp, Artificial Sequence
  Synthetic (ldhA down)
gtcacctcatcaagttctagaa

SEQ ID NO: 34 - DNA, 36 bp, Artificial Sequence
  Synthetic (0049-1 for)
gcaggcatgcaagcttaaagtccccacgggtctact

SEQ ID NO: 35 - DNA, 35 bp, Artificial Sequence
  Synthetic (0049-1 rev)
gagctcagtcagtcacggagaccacattgaggaca

SEQ ID NO: 36 - DNA, 35 bp, Artificial Sequence
  Synthetic (0049-2 for)
tgactgactgagctcccaaaatgcgcaacatcggc

SEQ ID NO: 37 - DNA, 36 bp, Artificial Sequence
  Synthetic (0049-2 rev)
ggccagtgccaagctttaccgatgtactgcacggag

SEQ ID NO: 38 - DNA, 36 bp, Artificial Sequence
  Synthetic (0463-1 for)
gcaggcatgcaagcttgcaccagaaggtgggctaaa

SEQ ID NO: 39 - DNA, 35 bp, Artificial Sequence
  Synthetic (0463-1 rev)
gagctcagtcagtcatgccgatagctctgcatctc

SEQ ID NO: 40 - DNA, 35 bp, Artificial Sequence
  Synthetic (0463-2 for)
tgactgactgagctcgtatgcatcgcagctaaccg

SEQ ID NO: 41 - DNA, 36 bp, Artificial Sequence
  Synthetic (0463-2 rev)
ggccagtgccaagctttacctgcagcattgtgcgca

SEQ ID NO: 42 - DNA, 36 bp, Artificial Sequence
  Synthetic (2619-1 for)
gcaggcatgcaagcttgtcttcctttaggtccgctg

SEQ ID NO: 43 - DNA, 35 bp, Artificial Sequence
  Synthetic (2619-1 rev)
gagctcagtcagtcaccggtgacaacctgcatcaa

SEQ ID NO: 44 - DNA, 35 bp, Artificial Sequence
  Synthetic (2619-2 for)
tgactgactgagctcggtgccggatttgagtggaa

SEQ ID NO: 45 - DNA, 36 bp, Artificial Sequence
  Synthetic (2619-2 rev)
ggccagtgccaagcttttgcccagctttagcgctgt

SEQ ID NO: 46 - DNA, 20 bp, Artificial Sequence
  Synthetic (0049for)
gtgatgctggtaaacctgtg

SEQ ID NO: 47 - DNA, 19 bp, Artificial Sequence
  Synthetic (0049rev)
ttgcgtttgggaaatacgg

SEQ ID NO: 48 - DNA, 20 bp, Artificial Sequence
  Synthetic (0463for)
gcaccagaaggtgggctaaa

SEQ ID NO: 49 - DNA, 20 bp, Artificial Sequence
  Synthetic (0463rev)
tacctgcagcattgtgcgca

SEQ ID NO: 50 - DNA, 20 bp, Artificial Sequence
  Synthetic (2619for)
gtcttcctttaggtccgctg

SEQ ID NO: 51 - DNA, 20 bp, Artificial Sequence
  Synthetic (2619rev)
ttgcccagctttagcgctgt

SEQ ID NO: 52 - DNA, 46 bp, Artificial Sequence
  Synthetic (MD-616)
aaagtgtaaagcctgggaacaacaagacccatcatagtttgccccc

SEQ ID NO: 53 - DNA, 36 bp, Artificial Sequence
  Synthetic (MD-618)
gttcttctaatcagaattggttaattggttgtaaca

SEQ ID NO: 54 - DNA, 40 bp, Artificial Sequence
  Synthetic (MD-615)
gcgtaatagcgaagaggggcgtttttccataggctccgcc

SEQ ID NO: 55 - DNA, 40 bp, Artificial Sequence
  Synthetic (MD-617)
gttcaatcataacaccccttgtattactgtttatgtaagc

SEQ ID NO: 56 - DNA, 31 bp, Artificial Sequence
  Synthetic (MD-619)
gggtgttatgattgaacaagatggattgcac

SEQ ID NO: 57 - DNA, 39 bp, Artificial Sequence
  Synthetic (MD-620)
attctgattagaagaactcgtcaagaaggcgatagaagg

SEQ ID NO: 58 - DNA, 17 bp, Artificial Sequence
  Synthetic (LacZa-NR)
cctcttcgctattacgc

SEQ ID NO: 59 - DNA, 21 bp, Artificial Sequence
  Synthetic (MD-404)
cccaggctttacactttatgc

SEQ ID NO: 60 - DNA, 47 bp, Artificial Sequence
  Synthetic (MD-627)
gccaccgcggtggagctcatttagcggatgattctcgttcaacttcg

SEQ ID NO: 61 - DNA, 32 bp, Artificial Sequence
  Synthetic (MD-628)
ttttatttgcaaaaacggccgaaaccatccct

SEQ ID NO: 62 - DNA, 40 bp, Artificial Sequence
  Synthetic (MD-629)
ccgtttttgcaaataaaacgaaaggctcagtcgaaagact

SEQ ID NO: 63 - DNA, 43 bp, Artificial Sequence
  Synthetic (MD-630)
gaacaaaagctggagctaccgtatctgtggggggatggcttgt

SEQ ID NO: 64 - DNA, 5342 bp, Artificial Sequence
  Synthetic (pGST1)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatccactagttctagagcggccgccaccgcggtggagctcatttagcggatgattctcgttcaacttcggccgaagccacttcgtctgtcataatgacagggatggtttcggccgtttttgcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc

SEQ ID NO: 65 - DNA, 5693 bp, Artificial Sequence
  Synthetic (gapA promoter-cat1-sucD-4hbD-cat2 cassette)
gtcgacatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaacatgtctaaaggaatcaagaatagccaattgaaaaaaaagaacgtcaaggccagtaacgttgctgagaagatcgaagagaaggtggaaaagaccgacaaggtcgttgagaaggctgctgaggtgaccgaaaagcgaattcgaaacttaaagctccaggaaaaagttgtgaccgcagatgtcgcagctgacatgatcgagaatggcatgatcgtcgcaattagcggcttcacgccatccgggtatccaaaggaggttccaaaagcccttactaagaaggttaatgcgctggaggaggagttcaaggtgacgctgtataccggttctagcacaggcgctgatattgacggagaatgggcgaaggcaggaataatcgaacggcgtatcccataccagaccaactctgacatgaggaaaaaaataaacgatggttcaatcaagtacgcagatatgcacctgagccatatggctcaatacattaactattctgtgattcctaaggttgacattgccatcatcgaggcggtggccattaccgaggaaggggatattattcctagtactggaatcggcaacacagctacgtttgtcgagaatgcggataaggtaattgtggaaataaacgaggctcagccgcttgagttggaaggcatggcagatatctataccctgaagaaccctccacgtcgcgagcccatcccgatagtcaacgcaggcaaccgcatagggaccacttacgtcacctgtggctctgaaaaaatctgcgcgatcgtcatgaccaacacccaagacaaaacccgcccactcaccgaagtttctcctgtcagtcaggcaatctccgataacctgattggcttcctgaacaaagaagtagaggagggtaaactcccaaaaaacctgctccccatacagtcaggtgtcggttcggttgctaacgccgtgctagccggactctgcgaatcaaacttcaaaaatttgagctgctacacagaagtgatccaggattcgatgttgaagcttatcaaatgtggaaaggcagatgtggtgtccggcacctcgatctcgccatcaccggaaatgctgcccgagttcataaaggacataaatttttttcgcgagaagatagtactgcgcccccaggaaatatctaataatccggaaatagctcgtcgtataggagtgatctccataaacactgctttggaagtagacatctacggtaatgtgaactccacgcatgtcatgggctccaagatgatgaacggcatcggcggcagcggcgactttgcccgcaacgcatacctcaccatattcactacggagtccatcgcgaagaagggcgacatttcctctatcgttcctatggtttcccacgtggaccacaccgagcatgacgtaatggtcatcgttaccgaacagggggttgcggatctccgcggtctttcccctcgggaaaaggccgtggcgataattgagaattgcgtccacccggattacaaggatatgctcatggagtacttcgaggaggcttgtaagtcctcaggtggcaacaccccacacaaccttgaaaaagccctatcctggcacactaagttcataaaaactggctcgatgaagtaattagaggagacacaacatggagattaaagagatggtcagtcttgcgcgcaaagctcagaaggagtatcaggccacccataaccaagaagctgtggacaacatctgccgagctgcagcgaaggttatttacgaaaatgcagcaattctggcccgcgaggcagtggacgaaaccggcatgggtgtttacgagcacaaggtggccaagaatcaaggcaagtccaaaggtgtttggtacaacctgcataacaagaagtcgattggcatcctcaatatcgatgagcgtaccggcatgatcgagatcgcaaaacctatcggggttgtaggcgccgttacgccaaccaccaaccctatcgttactccgatgagcaacatcatctttgctcttaagacctgcaacgccatcattatcgccccacacccgcgctccaaaaagtgctctgcccacgcagttcggctgatcaaagaggctatcgctccgttcaacgtgcccgaaggtatggttcagatcatcgaggagcctagcatcgagaagacgcaggaattgatgggcgccgtagacgtggtcgttgctaccgggggcatgggcatggtcaagtctgcctactcctcagggaagccttctttcggtgtcggagccggcaatgttcaggtgatagtggacagcaacatcgatttcgaagcggctgcagaaaagatcatcaccggacgtgccttcgacaacggtatcatctgctcaggcgaacagtccatcatctacaacgaggctgacaaggaagcagttttcacagcattccgcaaccacggtgcgtacttttgcgacgaggccgagggagatcgggctcgtgcagcgatcttcgaaaatggagccatcgcgaaagatgttgtgggccagtccgttgcctttattgccaagaaggcgaacattaatatccccgagggtactcgtattctcgtggtcgaagctcgcggagtaggcgccgaagatgtcatctgtaaagaaaagatgtgtccagtcatgtgcgccctctcctacaagcacttcgaagagggggtagagatcgcaaggacgaacctcgcaaacgaaggcaatggccatacctgtgctatccactccaacaaccaagcacacatcatcttggcaggctcggagctgaccgtgtctcgcatcgtggtcaacgcgccaagtgctaccacagcaggcggtcacatccagaacggtcttgccgtcaccaatactctaggctgcggctcttggggtaacaactcgatctccgaaaacttcacttataaacacctgctcaacatttcacgcatcgccccgttgaactccagcattcatatcccagatgataaggaaatctgggaactctaattagaggagacacaacatgcagcttttcaagctcaagagcgtcacacatcactttgatacttttgcagagtttgccaaggaattctgtctcggtgaacgcgacttggtaattaccaacgagttcatctacgaaccgtatatgaaggcatgccagctgccttgtcattttgtgatgcaggagaaatacggccaaggcgagccttctgacgagatgatgaacaacatcctagcagatatccgtaatatccagttcgaccgcgtgatcgggatcggaggtggtacggttattgacatctcaaaactctttgttctgaagggattaaatgatgttctcgacgcgttcgatcgcaagattccccttatcaaagagaaagaactgatcattgtgcccaccacctgcggaaccggctcggaggtgacgaacatttccatcgccgagatcaagtcccggcacaccaagatgggtttggctgacgatgcaattgttgctgaccacgccataatcatccctgaacttctgaagagcttgcccttccacttctatgcatgctccgcaatcgatgctcttattcatgccatcgagtcatacgtttctccaaaagcgtctccatactcccgtctgttcagtgaggcggcgtgggacattatcctggaagttttcaagaaaatcgccgaacacggcccagagtaccgcttcgagaagctgggggaaatgatcatggccagcaactatgccggtatcgctttcggcaacgcaggcgttggcgccgtccacgctctatcctacccgttgggcggcaactatcacgtgccgcatggagaagcaaactatcagttcttcaccgaggtctttaaagtataccaaaagaagaatccgttcggctatattgtcgaactcaactggaagctctccaagattctgaactgccagccagagtacgtgtacccgaagctggatgaactgctcggttgccttcttaccaagaaacctttgcacgaatacggcatgaaggacgaagaggttcgtggcttcgcggaatcggtcctgaagacccagcaacgcttgctcgccaacaactacgtcgaacttactgtcgatgagatcgaaggtatctaccgacgtctctactaattagaggagacacaacatgaaggatgtactggcggaatacgcctcccgcattgtttcggcggaggaggccgttaagcacatcaaaaacggtgaacgggtagctttgtcacacgctgccggcgtgcctcagagttgcgttgacgcactggtgcagcaggccgaccttttccagaatgtggaaatctatcacatgctgtgcctcggtgagggtaagtatatggcgcctgagatggcccctcacttccgccacatcaccaactttgtcggtggtaactcccgtaaggcggtcgaagaaaaccgggccgatttcattccggtattcttttacgaggtgccaagcatgattcgcaaagacatcctccacattgatgtcgccatcgttcagctttcaatgcctgacgaaaatggttactgttcctttggagtatcttgcgattactccaagccggcagcagagagcgctcacctggttatcggagaaatcaaccgtcaaatgccatacgtacacggcgacaacttgattcatatctccaagttggattacatcgtgatggcagactaccccatctactctcttgcaaagcccaagatcggggaagtcgaggaagctatcgggaggaattgtgccgagcttattgaagatggtgccactctccagctgggaatcggcgcgattcctgatgcggccctgttatttctcaaggacaaaaaggatctgggcatccataccgaaatgttctccgatggtgttgtcgaattggttcgctccggcgttatcacaggcaagaaaaagactcttcaccccggaaagatggtcgcaaccttcctgatgggaagcgaggacgtgtatcatttcatcgataaaaaccccgatgtagaactgtatccagtagattacgtgaatgacccgcgtgtgatcgcccaaaacgacaatatggtctcgattaacagctgcatcgaaatcgaccttatgggacaggtcgtgtccgagtgcatcggctcaaagcaattcagcggcaccggcggccaagttgactacgtgcgtggcgcagcatggtctaaaaacggcaaatcgatcatggcaatcccgtccactgcaaaaaacggtacggcatctcgaattgtacctatcatcgcggagggcgctgctgtcaccaccctgcgcaacgaggtcgattacgttgtaaccgagtacggtatcgctcagctcaagggcaagagcctgcgccagcgcgcagaggctttgatcgcgatagcccaccccgacttccgtgaggaactaacgaaacatctccgcaagcgattcggataagcggccgcagatctggatcc

SEQ ID NO: 66 - DNA, 44 bp, Artificial Sequence
  Synthetic (3G up)
gatatcgaattcctgcagatgattttgcatctgctgcgaaatct

SEQ ID NO: 67 - DNA, 47 bp, Artificial Sequence
  Synthetic (3G down)
gcggtggcggccgctctagattagtagagacgtcggtagataccttc

SEQ ID NO: 68 - DNA, 48 bp, Artificial Sequence
  Synthetic (ncgl0049_up F)
catgattacgccaagcttcaatgcctttgatttgaactaagcacagga

SEQ ID NO: 69 - DNA, 43 bp, Artificial Sequence
  Synthetic (ncgl0049_up R)
tgcaaaatcattagagctcaaaaccgcggcgtaaaatattaga

SEQ ID NO: 70 - DNA, 31 bp, Artificial Sequence
  Synthetic (ncgl 0049_down F)
tacggtgctagcatctgcccctttacaaatc

SEQ ID NO: 71 - DNA, 48 bp, Artificial Sequence
  Synthetic (ncgl0049_down R)
acgacggccagtgaattcggatcagccctaaatacaactatgagacag

SEQ ID NO: 72 - DNA, 42 bp, Artificial Sequence
  Synthetic (G3G F)
tgagctctaatgattttgcatctgctgcgaaatctttgtttc

SEQ ID NO: 73 - DNA, 32 bp, Artificial Sequence
  Synthetic (G3G R)
cagatgctagcaccgtatctgtggggggatgg

SEQ ID NO: 74 - DNA, 24 bp, Artificial Sequence
  Synthetic (cat1-seq1F)
agcccttactaagaaggttaatgc

SEQ ID NO: 75 - DNA, 22 bp, Artificial Sequence
  Synthetic (cat1-seq2R)
cgtacttgattgaaccatcgtt

SEQ ID NO: 76 - DNA, 22 bp, Artificial Sequence
  Synthetic (sucD-seq1F)
caatgttcaggtgatagtggac

SEQ ID NO: 77 - DNA, 20 bp, Artificial Sequence
  Synthetic (sucD-seq2R)
cacaacatctttcgcgatgg

SEQ ID NO: 78 - DNA, 21 bp, Artificial Sequence
  Synthetic (4hbD-seq1F)
ctgaccacgccataatcatcc

SEQ ID NO: 79 - DNA, 20 bp, Artificial Sequence
  Synthetic (4hbD-seq2R)
cgataccggcatagttgctg

SEQ ID NO: 80 - DNA, 5567 bp, Artificial Sequence
  Synthetic (pGSX1)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaacctcgaggtcgacggtatcgataagcttgatatcgaattcctgcagcccgggggatccactagttctagagcggccgccaccgcggtggagctcatttagcggatgattctcgttcaacttcggccgaagccacttcgtctgtcataatgacagggatggtttcggccgtttttgcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc

SEQ ID NO: 81 - DNA, 48 bp, Artificial Sequence
  Synthetic (Primer MD-625)
tagggcgaattgggtaccatgattttgcatctgctgcgaaatctttgt

SEQ ID NO: 82 - DNA, 55 bp, Artificial Sequence
  Synthetic (Primer MD-626)
gataccgtcgacctcgaggttgtgtctcctctaaagattgtaggaaatgcaatgt

SEQ ID NO: 83 - DNA, 34 bp, Artificial Sequence
  Synthetic (Primer 1502-up)
agaggagacacaaccatgactgaagtagcaaccg

SEQ ID NO: 84 - DNA, 34 bp, Artificial Sequence
  Synthetic (Primer 1502-dn)
tggcggccgctctagttacatgccaagaactgcg

SEQ ID NO: 85 - DNA, 34 bp, Artificial Sequence
  Synthetic (Primer 1503-up)
agaggagacacaacccggatcatgacttcggcaa

SEQ ID NO: 86 - DNA, 33 bp, Artificial Sequence
  Synthetic (Primer 1503-dn)
tggcggccgctctagttagcccaccgatccttc

SEQ ID NO: 87 - DNA, 35 bp, Artificial Sequence
  Synthetic (Primer 0776-up)
agaggagacacaaccatggtgaaaaaccgattcaa

SEQ ID NO: 88 - DNA, 33 bp, Artificial Sequence
  Synthetic (Primer 0776-dn)
tggcggccgctctagctactgagctgccttctc

SEQ ID NO: 89 - DNA, 945 bp, Corynebacterum glutamicum
atgaaagaaaccgtcggtaacaagattgtcctcattggcgcaggagatgttggagttgcatacgcatacgcactgatcaaccagggcatggcagatcaccttgcgatcatcgacatcgatgaaaagaaactcgaaggcaacgtcatggacttaaaccatggtgttgtgtgggccgattcccgcacccgcgtcaccaagggcacctacgctgactgcgaagacgcagccatggttgtcatttgtgccggcgcagcccaaaagccaggcgagacccgcctccagctggtggacaaaaacgtcaagattatgaaatccatcgtcggcgatgtcatggacagcggattcgacggcatcttcctcgtggcgtccaacccagtggatatcctgacctacgcagtgtggaaattctccggcttggaatggaaccgcgtgatcggctccggaactgtcctggactccgctcgattccgctacatgctgggcgaactctacgaagtggcaccaagctccgtccacgcctacatcatcggcgaacacggcgacactgaacttccagtcctgtcctccgcgaccatcgcaggcgtatcgcttagccgaatgctggacaaagacccagagcttgagggccgtctagagaaaattttcgaagacacccgcgacgctgcctatcacattatcgacgccaagggctccacttcctacggcatcggcatgggtcttgctcgcatcacccgcgcaatcctgcagaaccaagacgttgcagtcccagtctctgcactgctccacggtgaatacggtgaggaagacatctacatcggcaccccagctgtggtgaaccgccgaggcatccgccgcgttgtcgaactagaaatcaccgaccacgagatggaacgcttcaagcattccgcaaataccctgcgcgaaattcagaagcagttcttctaa

SEQ ID NO: 90 - DNA, 4990 bp, Artificial Sequence
  Synthetic (pGSK+)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc